(19) United States
(12) Patent Application Publication
(10) Pub. No.: US 2011/0033874 A1
(43) Pub. Date: Feb. 10, 2011

(54) BIOMARKERS

(75) Inventors: Christopher Joseph Pemberton, Christchurch (NZ); Arthur Mark Richards, Christchurch (NZ); Michael Gary Nicholls, Christchurch (NZ); Timothy Grant Yandle, Christchurch (NZ)

Correspondence Address: DUANE MORRIS LLP - San Diego, 101 WEST BROADWAY, SUITE 900, SAN DIEGO, CA, 92101-8285, US

(73) Assignee: OTAGE INNOVATION LIMITED, Dunedin (NZ)

(21) Appl. No.: 12/919,002

(22) PCT Filed: Feb. 20, 2009
(86) PCT No.: PCT/NZ2009/000022

§ 371 Date: Aug. 23, 2010

Related U.S. Application Data
(60) Provisional application No. 61/030,629, filed on Feb. 22, 2008.

(51) Int. Cl. G01N 33/53; C07K 16/18; C07H 21/04; C12N 15/63; C12N 5/10; C07K 7/06; C07K 2/00; C12P 21/02

(52) U.S. Cl. 435/7.21; 530/387.9; 530/387.3; 436/501; 435/7.92; 536/23.1; 435/320.1; 435/325; 530/300; 530/328; 435/69.1

(57) ABSTRACT

The invention provides methods for predicting, diagnosing or monitoring acute cardiac disorders, cardiac transplant rejection, or distinguishing acute cardiac disorders from pulmonary disorders, by measuring ANP signal peptide levels in a sample taken from a subject shortly after onset of, or presentation with the disorder or transplant rejection. Also provided are antibodies useful in the methods of the invention.

This application is a US national stage of PCT/NZ/2009/000022 filed on Feb. 20, 2009 which claims the benefit of U.S. Provisional Application Ser. 61/030,629 filed on Feb. 22, 2008; the contents of each of which is incorporated herein by reference in its entirety.

### FIELD OF THE INVENTION

This invention relates to ANP signal peptide (ANP-SP) and its use in the prognosis, diagnosis and monitoring of acute cardiac disorders including acute coronary syndromes in a subject resulting in release of biomarker into the circulation. More particularly, the invention relates to methods of predicting, diagnosing or monitoring an acute cardiac disorder in a subject by measuring ANP-SP levels in a sample taken shortly after onset of, or at clinical presentation with the disorder.

### BACKGROUND

Acute cardiac disorders including acute coronary syndromes (ACS) encompass a wide spectrum of cardiac ischemic events ranging from unstable angina through to acute myocardial infarction (AMI). AMI presents as the most serious of these events and therefore requires rapid and accurate diagnosis. Patients who present with two or more of the described features (a history of ischemic chest discomfort, evolutionary changes on serial electrocardiogram (ECG) traces and a rise and fall in plasma cardiac biomarkers) are clearly identified as undergoing AMI.²⁵ However, a significant proportion of patients (40%-50%) who present with suspected AMI do not have serial changes on ECG, or typical symptoms thus placing heavy emphasis on circulating biomarker concentrations for accurate diagnosis.^{25,26}

Accurate early diagnosis of myocardial infarction facilitates prompt introduction of reperfusion treatment, including effective percutaneous or thrombolytic revascularisation and adjunctive anticoagulant and anti-platelet therapy. Such treatments are progressively less effective at reducing mortality and morbidity with each hour of delay in diagnosis and management.²⁻⁴ Given the need for accelerated decision-making in this clinical situation, there is considerable interest in the identification of circulating biomarkers providing an early and specific diagnosis of acute cardiac disorders, particularly AMI.

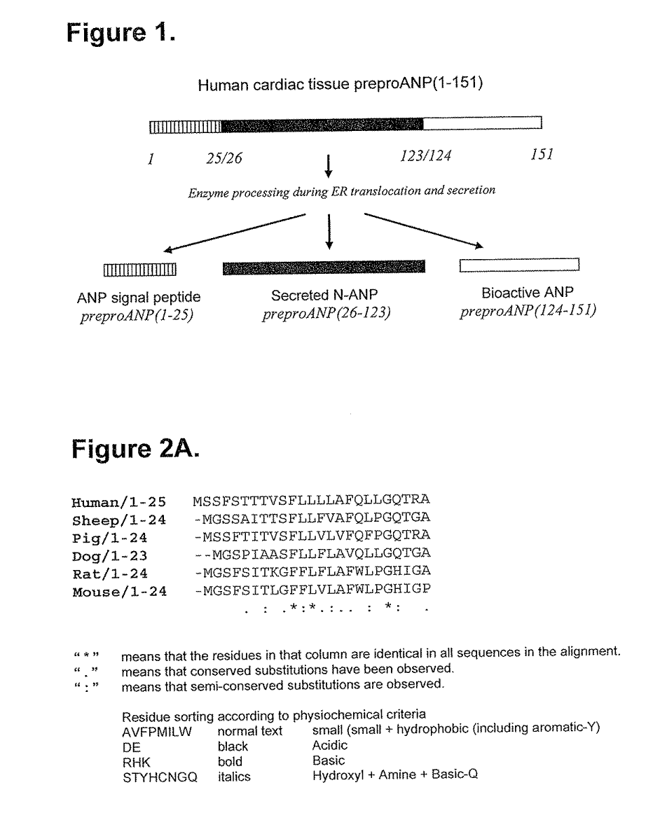

Indeed current clinical guidelines highlight the importance of biomarker measurement in the identification of myocardial infarction and acute coronary syndromes.²⁵ A number of biomarkers have been proposed for this purpose, including creatine kinase-MB (CK-MB), troponin T (TnT), troponin I (TnI) and myoglobin, but there are limitations to their use. Time to detectable or abnormal elevation of plasma cardiac biomarkers can be 6 hours (myoglobin, CK-MB) to 12 hours (TnT, Trd) with peak levels not occurring until 24-48 hours after onset of injury, imposing a window of delay upon precise diagnosis and treatment.¹⁻⁴ Furthermore, both myoglobin and CK-MB are non-specific and can be secreted from extra-cardiac sources, especially during trauma or surgery.¹ Other biomarkers useful for this purpose are ANP (preproANP (124-151)), N-ANP (preproANP 26-123)) (see FIG. 1), BNP (preproBNP 103-134) and N-BNP (preproBNP (27-134) which is also known as NT-proBNP. These peptides are secreted into the circulation.⁶

Measurement of plasma concentrations of ANP and N-ANP early post-AMI has powerful prognostic value^{2,3,7} and incorporation of plasma concentrations of these peptides into treatment regimes may improve clinical outcomes of patients with heart failure.⁸ This is particularly true of N-ANP which has a half-life some 20-fold longer than ANP⁵ and thus provides additional important information regarding long term cardiac performance after AMI.

As with the cardiac biomarkers above, ANP and N-ANP may not reach detectable or abnormal levels for 6 to 12 hours after onset of injury, with peak levels not occurring until 24 to 48 hours after onset. The long term diagnostic/predictive powers of ANP and N-ANP therefore lack the accompanying power of a specific marker providing early specific diagnosis of acute cardiac disorders such as acute cardiac injury within the first few hours of clinical presentation. A need thereof exists for such an early marker.

More recently, it has been suggested that ANP-SP and BNP-SP may be useful in diagnosing heart disease (US 2005/0244904, WO 2005/052593). It is generally indicated that levels of ANP-SP and BNP-SP will be higher in heart failure patients than normal patients. No time course information as to when to measure ANP-SP or BNP-SP levels is provided. It is stated that BNP-SP levels are elevated in conjunction with N-BNP.

It is an object of the present invention to go some way towards fulfilling the need for an early marker of acute cardiac disorders, and/or to at least provide the public with a useful choice.

### SUMMARY OF THE INVENTION

Human atrial natriuretic signal peptide (ANP-SP) or preproANP (1-25) is a 25 amino acid peptide cleaved from preproANP (1-151) SEQ ID NO:1. ANP-SP (1-25) is shown separately in SEQ ID NO:14.

The applicants have surprisingly discovered that the circulating concentration of ANP-SP is highest in the first few hours following onset of, or at clinical presentation with suspected acute coronary syndromes (ACS). Peaks are in the order of five to fifteen, commonly three to seven times higher than normal control populations in these first hours.

Accordingly, in a first aspect the present invention provides a method for predicting, diagnosing or monitoring an acute cardiac disorder (ACD) in a subject, the method comprising: measuring the level of ANP-SP in a biological sample obtained from the subject within four hours of onset of the ACD, or within four hours of presentation with the ACD; and comparing the level of said ANP-SP with the ANP-SP level from a control wherein a measured level of ANP-SP higher than the control level is indicative of ACD.

The invention also provides a method for monitoring a response to treatment of an acute cardiac disorder (ACD) in a subject, the method comprising measuring the level of ANP-SP in a biological sample obtained from the subject within four hours of onset of the ACD or within four hours of presentation with the ACD; and comparing the level of said ANP-SP with the ANP-SP level from a control, wherein a change in the measured level of ANP-SP from the control level is indicative of a response to the treatment.

In another aspect, the invention also provides a method for predicting, diagnosing or monitoring a cardiac transplant rejection episode in a subject, the method comprising measuring the level of ANP-SP in a biological sample obtained from a subject within four hours of heart transplant and comparing the level of said ANP-SP with the ANP-SP level from a control, wherein a measured level of ANP-SP higher than the control level is indicative of transplant rejection.

The invention also provides a method of distinguishing between a pulmonary disorder and an acute cardiac disorder (ACD) in a subject, the method comprising measuring the level of ANP-SP in a biological sample obtained from a subject within four hours of presentation with the disorder; and comparing the level of said ANP-SP with the ANP-SP level from a control wherein a measured level of ANP-SP higher than the control level is indicative of ACD.

The invention also provides a method for predicting, diagnosing or monitoring an acute cardiac disorder (ACD), cardiac transplant rejection, or ACD/pulmonary disorder in a subject, the method comprising measuring the level of ANP-SP in a biological sample obtained from the subject within the first four hours of onset of, or clinical presentation with ACD, cardiac transplant rejection or ACD/pulmonary disorder, wherein a measured level of ANP-SP is compared with the ANP-SP level from a control wherein a measured level of ANP-SP higher than the control level is indicative of ACD or cardiac transplant rejection.

In one embodiment, the methods of the invention are in vitro methods.

In one embodiment, the measurement of ANP-SP levels is carried out within two hours, or one hour, or within 30 minutes of onset or clinical presentation.

In one embodiment, the biological sample is blood, saliva, interstitial fluid, plasma, urine, serum or heart tissue.

In one embodiment, the measuring step comprises
(a) binding ANP-SP with a binding agent; and
(b) measuring the level of bound ANP-SP.

The binding agent in one embodiment is an antibody or antibody fragment. Most commonly, the antibody is a monoclonal, polyclonal, chimeric or humanized antibody. In one embodiment the antibody is a monoclonal antibody.

In an alternate embodiment, the levels of ANP-SP are measured using mass spectroscopy.

The ANP-SP which is bound by the antibody is the full length human ANP-SP molecule (SEQ ID NO:14) or a variant or fragment thereof. In one embodiment binding is selective. In one embodiment, the fragment is at least four contiguous amino acids in length. Desirably, the antibody binds the N-terminus or the C-terminus of ANP-SP.

Specific antigenic peptides which the binding agent binds or selectively binds include human ANP-SP (16-25) (SEQ ID NO:12), human ANP-SP (1-10) (SEQ ID NO:16), or antigenic-binding fragments or variants thereof.

Binding of ANP-SP in one embodiment is measured using antibodies or antibody fragments that are immobilised on a solid phase.

Levels of ANP-SP may usefully be measured with an assay selected from RIA, ELISA, fluoroimmunoassay, immunofluorometric assay, mass spectrometry and immunoradiometric assay.

Accordingly, the invention also provides an assay for ANP-SP in a biological sample obtained from a subject within four hours from onset of, or within four hours of clinical presentation with ACD, cardiac transplant rejection, or ACD/pulmonary disorder, the assay comprising detecting and measuring the level of ANP-SP in the sample using any known methods.

The invention also provides an assay for ANP-SP comprising:
(a) binding one or more ANP-SP polypeptides from a biological sample, wherein the ANP-SP polypeptide is selected from the group ANP-SP 1-10 (SEQ ID NO:16), and ANP-SP 16-25 (SEQ ID NO:12), or a variant or fragment thereof; and
(b) measuring the level of bound ANP-SP polypeptide.

In one embodiment, the assay is an in vitro assay.

The methods of the invention may further comprise measuring the level of one or more non-ANP-SP markers of said ACD, or cardiac transplant rejection, or ACD/pulmonary disorder and comparing the levels against marker levels from a control wherein a deviation in the measured level from the control level of non-ANP-SP marker, together with a measured level of ANP-SP which is higher than the control level of ANP-SP, is predictive or diagnostic of the ACD, or can be used to monitor said. ACD, cardiac transplant rejection or ACD/pulmonary disorder.

Markers for use in the context of acute coronary syndrome include troponin T, troponin I, creatine kinase MB, myoglobin, BNP, BNP-SP, NT-BNP, LDH, aspartate aminotransferase, and heart specific fatty acid binding protein (H-FABP).

In another aspect, the present invention also provides an ANP-SP binding agent that binds or selectively binds ANP-SP (SEQ ID NO:14) or an antigenic-binding fragment or variant thereof for use in predicting, diagnosing or monitoring an acute cardiac disorder (ACD), cardiac transplant rejection or ACD/pulmonary disorder in a subject, wherein the ACD, cardiac transplant rejection or ACD/pulmonary disorder is characterised by the appearance of ANP-SP in a biological sample obtained from the subject within four hours of onset of, or within four hours of clinical presentation with ACD, cardiac transplant rejection or ACD/pulmonary disorder.

In one embodiment, the binding agent is an antibody or antigen-binding fragment thereof.

The invention also provides an antibody or antigen-binding fragment thereof which binds
(a) ANP-SP amino acid sequence 16-25 (SEQ ID NO:12) or 1-10 (SEQ ID NO:16);
(b) an amino acid sequence encoded by a nucleotide sequence selected from SEQ ID NO:13 or SEQ ID NO:17; or
(c) a variant or fragment of (a) or (b).

The antibody may be a monoclonal, polyclonal, chimeric or humanized antibody.

The invention is also directed to the use of an ANP-SP binding agent in the manufacture of a prognostic, diagnostic or monitoring tool for assessing an acute cardiac disorder (ACD), cardiac transplant rejection or ACD/pulmonary disorder in a subject, wherein assessment is carried out within four hours of onset of, or within four hours of clinical presentation with ACD, cardiac transplant rejection or ACD/pulmonary disorder.

The invention also relates to the use of an antibody or antigen binding fragment of the invention in the manufacture of a prognostic, diagnostic or monitoring tool for assessing an acute cardiac disorder (ACD), cardiac transplant rejection or ACD/pulmonary disorder in a subject.

The invention also relates to a use of the invention wherein the prognostic, diagnostic or monitoring tool is calibrated to measure ANP-SP levels in the range of from 0.1 to 500 pmol/L, or 1 to 300 pmol/ L, or 2 to 100 pmol/L, or 5 to 150 pmol/L.

In another aspect, the invention provides a kit for predicting, diagnosing or monitoring an acute cardiac disorder (ACD), cardiac transplant rejection or ACD/pulmonary disorder comprising an ANP-SP binding agent, wherein the kit is for use with a biological sample obtained from a subject within four hours of onset of, or clinical presentation with ACD, cardiac transplant rejection or ACD/pulmonary disorder.

The invention also provides a kit for predicting, diagnosing or monitoring an acute cardiac disorder (ACD), cardiac transplant rejection or ACD/pulmonary disorder comprising an antibody or antigen-binding fragment of the invention.

The invention also provides a kit for predicting, diagnosing or monitoring an acute cardiac disorder (ACD) comprising a binding agent or antibody or antigen-binding fragment of the invention. In one embodiment, the kit is calibrated to measure ANP-SP levels in the range of 0.1 to 500 pmol/L, or 1 to 300 pmol/L, or 2 to 100 pmol/L, or 5 to 150 pmol/L.

In one embodiment, the kit also includes instructions for predicting, diagnosing or monitoring ACD, cardiac transplant rejection, or ACD/pulmonary disorder in a subject within four hours of onset, or clinical presentation, from the ANP-SP level measured in the biological sample obtained within four hours of onset or clinical presentation, and comparing the measured level to a control level. A measured ANP-SP level higher than the control level is indicative of ACD or transplant rejection.

In another aspect, the invention relates to nucleic acid molecule encoding an ANP-SP (16-25) (SEQ ID NO:12) or ANP-SP (1-10) (SEQ ID NO:16) wherein said nucleic acid is
(a) SEQ ID NO:13 or SEQ ID NO:17 or a variant or fragment thereof;
(b) a sequence which has 70%, 75%, 80%, 90%, 95% or 99% sequence identity to SEQ ID NO:13 or SEQ ID NO:17;
(c) a sequence which hybridises under stringent conditions to SEQ ID NO:13 or SEQ ID NO:17 or a fragment or variant thereof;
(d) a sequence of at least 10 nucleotides in length, capable of hybridising to the sequence of any one of (a) to (c) under stringent conditions;
(e) a complement of any one of (a) to (d);
with the proviso that the sequence is not SEQ ID NO: 15.

The invention also provides a genetic construct comprising a nucleic acid molecule of the invention, a vector comprising the genetic construct, a host cell comprising the genetic construct or vector, a polypeptide encoded by a nucleic acid molecule of the invention, an antibody which selectively binds a polypeptide of the invention, and a method for recombinantly producing a polypeptide of the invention.

Accordingly, in another aspect the invention provides an ANP-SP polypeptide or a variant or fragment thereof selected from:
(a) ANP-SP (16-25) (SEQ ID NO:12) or variant or fragment thereof;
(b) ANP-SP (1-10) (SEQ ID NO:16) or a variant or fragment thereof; or
(c) an amino acid sequence having at least 70%, 75%, 80%, 85%, 90%, 95% or 99% amino acid identity to the polypeptide of SEQ ID NO:12 or SEQ ID NO:16.

### BRIEF DESCRIPTION OF THE DRAWINGS

The invention will now be described with reference to the figures in the accompanying drawings in which

### FIGURES

FIG. 1 is a schematic diagram outlining the processing of human preproANP resulting in generation of free Signal, N-ANP and ANP peptides;

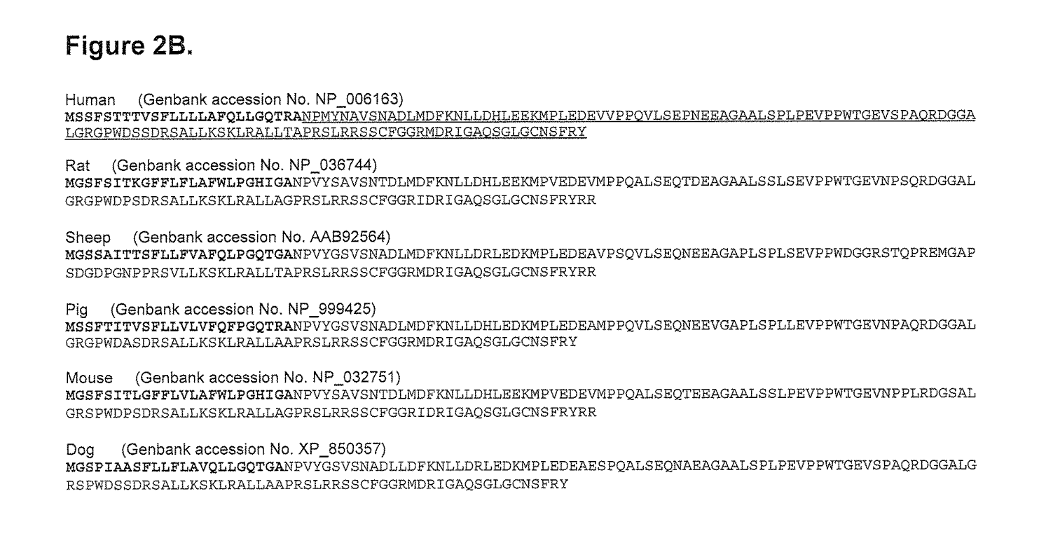

FIG. 2A is a Clustal W version 1.83 JALVIEW multiple sequence alignment of the preproANP signal peptide sequences in six species. The default Clustal W parameters were used in this alignment as follows: DNA Gap Open Penalty=15.0; DNA Gap Extension Penalty=6.66; DNA matrix=identity; Protein Gap Open Penalty=10.0; Protein Gap Extension Penalty=0.2; Protein Matrix=Gonnet; Protein/DNA ENDGAP=−1; Protein/DNA GAPDIST=4. The amino acids were submitted in the Pearson (fasta) format.⁹

FIG. 2B is a single letter notation format of preproANP sequences in six species. The signal peptide region is in bold;

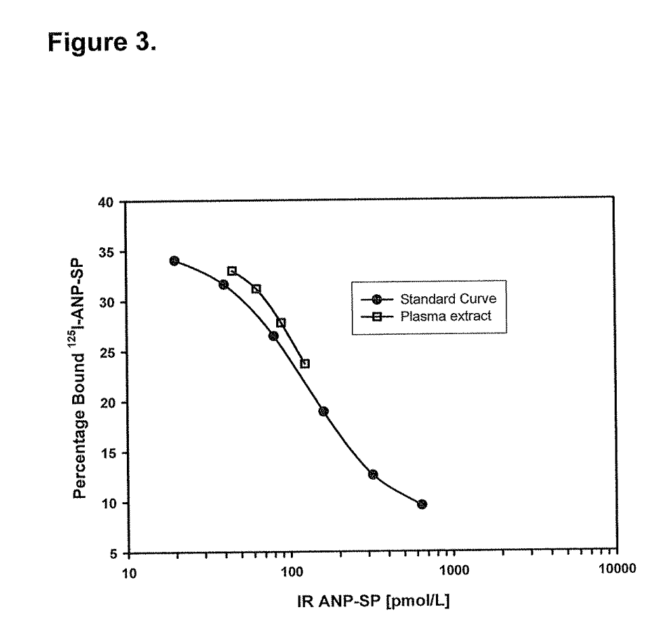

FIG. 3 shows the results of a radioimmunoassay with human plasma extracts (open squares) dilute in parallel with the ANP-SP standard curve (filled circles);

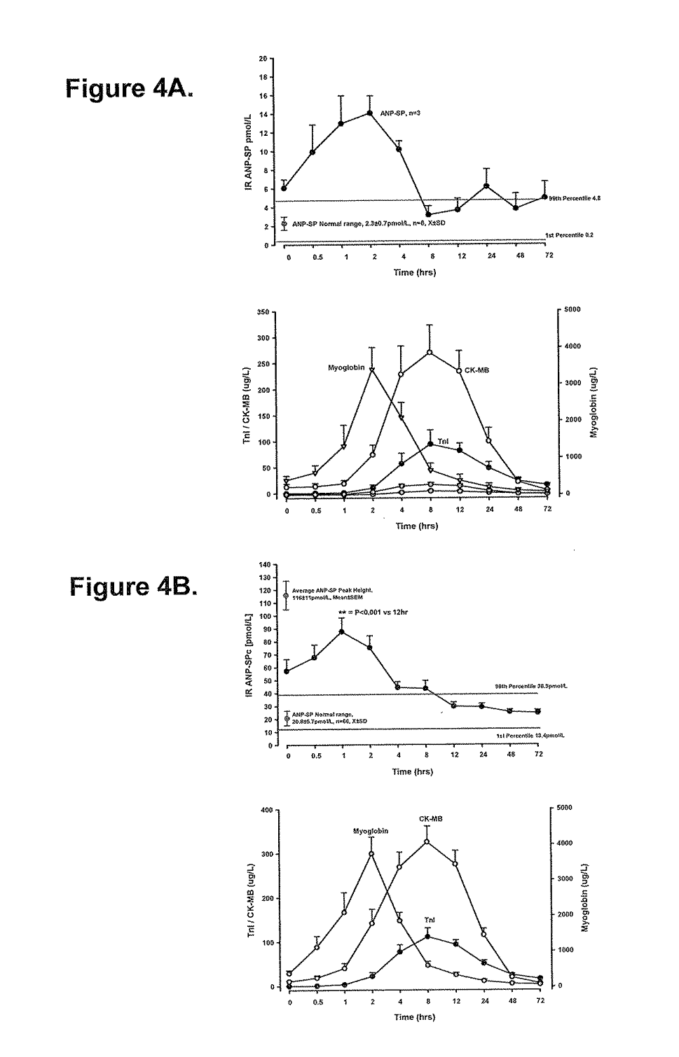

FIG. 4A Radioimmunoassay results showing Upper panel: Concentrations of ANP-SP in plasma drawn from AMI patients (n=3) at the times shown from hospital admission. Highest levels of ANP-SP were seen at 1-2 hours post-admission, being some 6 to 7-fold higher on average than levels measured in normal healthy individuals (filled lower circle, n=8, percentile ranges shown). Lower panel: matched, time course concentration profiles of CK-MB, myoglobin and TnT in the same patients in upper panel;

FIG. 4B Radioimmunoassay results showing Upper panel: Concentrations of ANP-SP in plasma drawn from AMI patients (n=23) at the times shown from hospital admission. Highest levels of ANP-SP were seen at 1-2 hours post-admission, being some 5 to 7-fold higher on average than levels measured in normal healthy individuals (filled lower circle, n=66, percentile ranges shown). Lower panel: matched, time course concentration profiles of CK-MB, myglobin and TnT in the same patients in upper panel.

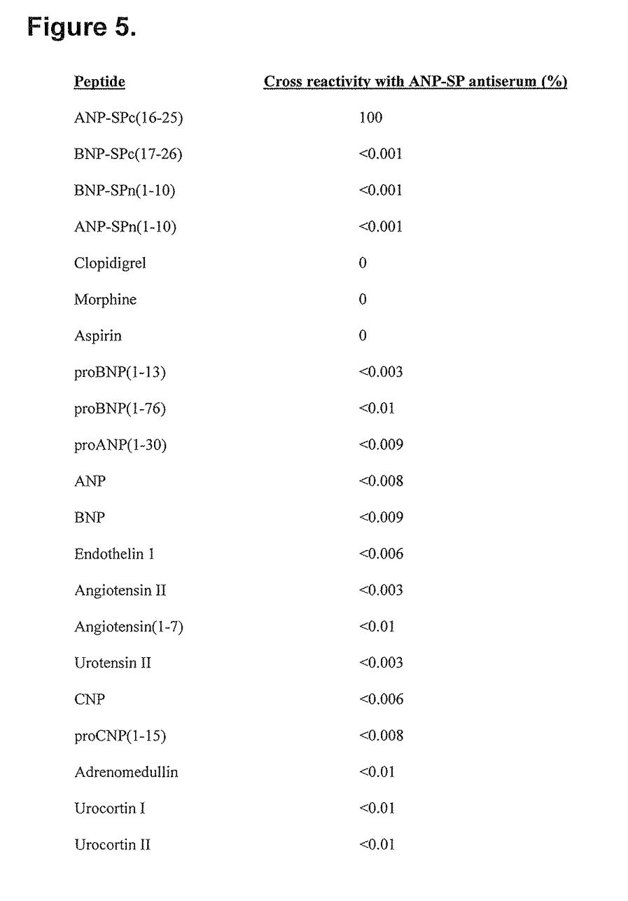

FIG. 5 Shows a table of cross reactivity data of ANP-SP antiserum; and

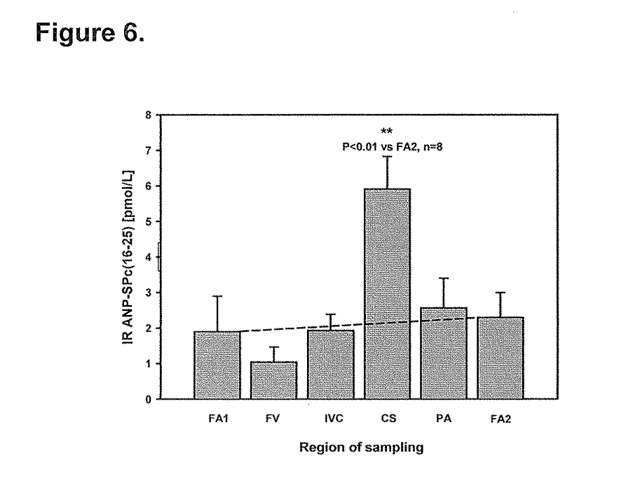

FIG. 6 is a bar graph showing circulating ANP-SP concentrations in patients are derived from a cardiac source.

### DEFINITIONS

Acute Cardiac Disorder (ACD), includes but is not limited to: acute coronary syndromes: (AMI) with ST-elevation on presenting ECG, unstable angina, and acute non ST-elevated MI; cardiac ischemia; acute cardiac injury; acute cardiac damage resulting from acute drug toxicity, acute cardiomyopathies, and cardiac transplant rejection. Full descriptive, definitions of these disorders are found in reference 1.

ACD/pulmonary disorder refers to a subject with an undiagnosed, or suspected ACD or pulmonary disorder.

Acute coronary syndromes (ACS) encompasses a wide spectrum of cardiac ischemia events including unstable angina, acute myocardial infarct with ST-elevation on presenting electrocardiogram (ECG), and acute myocardial infarction without ST-segment elevation on ECG.

The term “antibody” refers to an immunoglobulin molecule having a specific structure that interacts (binds) specifically with a molecule comprising the antigen used for synthesizing the antibody or with an antigen closely related to it. As used herein, the term “antibody” broadly includes full length antibodies and may also include certain antibody fragments thereof. Also included are monoclonal and polyclonal antibodies, multivalent and monovalent antibodies, multispecific antibodies (for example bi-specific antibodies), chimeric antibodies, human antibodies, humanized antibodies and antibodies that have been affinity matured. An antibody binds selectively or specifically to a ANP-SP polypeptide of the invention if the antibody binds preferentially to the ANP-SP e.g. has less than 25%, or less than 10%, or less than 1% or less than 0.1% cross-reactivity with a non-ANP-SP polypeptide. Usually, the antibody will have a binding affinity (dissociation constant (Kd) value), for the antigen or epitope of no more than 10⁻⁶, or 10⁻⁷M, or less than about 10⁻⁸M, ort 10⁻⁹M, or 10⁻¹⁰, or 10⁻¹¹ or 10⁻¹²M. Binding affinity may be assessed using surface plasma resonance.

As used herein, an “antigen-binding fragment” or “antibody fragment” means a portion of the intact antibody that preferably retains most or all, or minimally at least one of, the normal functions of that antibody fragment. An antibody fragment, for example, may comprise an Fc region that retains all or most or some of the function of the corresponding Fc region in the intact antibody. Examples of antibody fragments include Fab, Fab′, F(ab′)₂ and Fv fragments, linear antibodies, diabodies, single chain antibodies (ScFV) and multispecific antibodies.

As used herein, a “monoclonal antibody” means an antibody that is a highly specific antibody directed against a single target antigen. A monoclonal antibody may be obtained from a population of homogenous or substantially homogenous antibodies wherein each monoclonal antibody is identical and/or bind the same epitope, except for natural mutations which may occur in minor amounts.

An “isolated antibody” is an identified antibody which has been separated or recovered, or both, from a component of its natural environment. For example, separated from proteins including enzymes and hormones. In one embodiment, the antibody is purified to at least 95%, or 96% or 97% or 98% or 99% by weight of antibody. Purity can be determined by the Lowry method for example. Ordinarily the antibody will be prepared by at least one purification step.

The term “binding agent” as used herein refers to any solid or non-solid material capable of binding ANP-SP or a fragment or variant thereof. In one embodiment the term refers to any natural or non-natural molecule that binds to ANP-SP or a fragment or variant thereof. Examples of binding agents include proteins, peptides, nucleic acids, carbohydrates, lipids, and small molecule compounds. A selective or specific binding agent is an antibody or antigen-binding fragment thereof.

Biological sample as used herein means any sample derived from a subject to be screened. The sample may be any sample known in the art in which the ANP-SP can be detected. Included are any body fluids such as plasma, blood, saliva, interstitial fluid, serum, urine, synovial, cerebrospinal, lymph, seminal, amniotic, pericardial fluid and ascites, as well as tissues such as cardiac tissues but not limited thereto.

The term “epitope” includes any protein determinant capable of specific binding to an immunoglobulin and/or T cell receptor. That is, a site on an antigen to which B and/or T cells respond. Epitopic determinants usually consist of chemically active surface groupings of molecules such as amino acids or sugar side chains, and usually have specific three dimensional structural characteristics, and specific charge characteristics. An epitope typically includes 3, 5 or usually 8-10 amino acids. The amino acids may be contiguous, or non-contiguous amino acids juxtaposed by tertiary folding.

The term “within four hours of onset or clinical presentation” includes from 1 minute up to and including 240 minutes from onset of, or presentation at a medical facility with ACD, cardiac transplant rejection or an undiagnosed or suspected ACD/pulmonary disorder. Preferably measurements are made within 2 hours (from 1 minute up to and including 120 minutes) or within 1 hour (from 1 minute up to and including 60 minutes) from onset or presentation, within 5 to 45 minutes, 15 to 40 minutes, 20 to 35 minutes, or within 25 to 30 minutes of onset or presentation.

A level “higher” or “lower” than a control, or a change or deviation from a control in one embodiment is statistically significant. A higher level, lower level, deviation from, or change from a control level or mean control level can be considered to exist if the level differs from the control level by 5% or more, by 10% or more, by 20% or more, or by 50% or more compared to the control level. Statistically significant may alternatively be calculated as P<0.05. In a further alternative, higher levels, lower levels, deviation, and changes can be determined by recourse to assay reference limits or reference intervals. These can be calculated from intuitive assessment or non-parametric methods. Overall, these methods calculate the 0.025, and 0.975 fractiles as 0.025*(n+1) and 0.975 (n+1). Such methods are well known in the art. ^{22,23} Presence of a marker absent in a control, is also contemplated as a higher level, deviation or change. Absence of a marker present in a control is also contemplated as a lower level, deviation or change.

Included are samples from any subjects such as from normal healthy subjects with no clinical history of ACD and subjects with various ACD's including but not limited to acute coronary syndromes: (AMI) with ST-elevation on presenting ECG, unstable angina, and acute non ST-elevated. MI; cardiac ischemia; acute cardiac injury; acute cardiac damage resulting from acute drug toxicity, acute cardiomyopathies, and cardiac transplant rejection.

The term ANP-SP refers to the complete 25 amino acid ANP signal peptide for the human prepro ANP sequence (SEQ ID NO: 1). ANP-SP (1-25) is shown separately in SEQ ID NO:14 and underlined in FIG. 2B. Also encompassed within the term ANP-SP is a variant or fragment of ANP-SP. In one embodiment ANP-SP functions as a signal polypeptide, or as an antigenic polypeptide to which an antibody can bind. Variants and fragments of ANP-SP include variants and fragments which retain either or both of these functions.

The term “cardiomyopathies” as used herein refers to diseases of the myocardium where the myocardium or heart muscle is weakened. This can result in reduced pumping of the heart. Common causes of cardiomyopathies are heart attacks, viral infections, high blood pressure, alcoholism, and autoimmune diseases.

The term “comprising” as used in this specification and claims means “consisting at least in part of”; that is to say when interpreting statements in this specification and claims which include “comprising”, the features prefaced by this term in each statement all need to be present but other features can also be present. Related terms such as “comprise” and “comprised” are to be interpreted in similar manner.

The term “polynucleotide(s),” as used herein, means a single or double-stranded deoxyribonucleotide or ribonucleotide polymer of any length, and include as non-limiting examples, coding and non-coding sequences of a gene, sense and antisense sequences, exons, introns, genomic DNA, cDNA, pre-mRNA, mRNA, rRNA, siRNA, miRNA, tRNA, ribozymes, recombinant polynucleotides, isolated and purified naturally occurring DNA or RNA sequences, synthetic RNA and DNA sequences, nucleic acid probes, primers, fragments, genetic constructs, vectors and modified polynucleotides. Reference to a nucleic acid molecule is to be similarly understood.

A “fragment” of a polynucleotide sequence provided herein is a subsequence of contiguous nucleotides that is capable of specific hybridization to a target of interest, e.g., a sequence that is at least 10 nucleotides in length. The fragments of the invention comprise 10, 15 16, 17, 18, 19, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, or 74, contiguous nucleotides of a polynucleotide of SEQ ID NO:15. A fragment of a polynucleotide sequence can be used as a primer, a probe, included in a microarray, or used in polynucleotide-based selection methods herein. Fragments of other polynucleotides of the invention (such as SEQ ID NO:13 or SEQ ID NO:17) or polynucleotides described herein should be similarly understood.

The term “primer” refers to a short polynucleotide, usually having a free 3′OH group, that is hybridized to a template and used for priming polymerization of a polynucleotide complementary to the target.

The term “probe” refers to a short polynucleotide that is used to detect a polynucleotide sequence, that is complementary to the probe, in a hybridization-based assay. The probe may consist of a “fragment” of a polynucleotide as defined herein.

The term “polypeptide”, as used herein, encompasses amino acid chains of any length, but commonly at least 4 amino acids, at least 5 amino acids, or at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, or all 25 amino acids of the full-length ANP-SP protein (SEQ ID NO:14), in which amino acid residues are linked by covalent peptide bonds. Polypeptides useful in the present invention may be purified natural products, or may be produced partially or wholly using recombinant or synthetic techniques. The term may refer to a polypeptide, an aggregate of a polypeptide such as a dimer or other multimer, a fusion polypeptide, a polypeptide fragment, a polypeptide variant, or derivative thereof. Reference to other polypeptides of the invention (such as SEQ ID NO:12 or SEQ ID NO 16) or other polypeptides described herein should be similarly understood.

A “fragment” of a polypeptide is a subsequence of the polypeptide that performs a function that is required for the biological activity or binding and/or provides three dimensional structure of the polypeptide. The term may refer to a polypeptide, an aggregate of a polypeptide such as a dimer or other multimer, a fusion polypeptide, a polypeptide fragment, a polypeptide variant, or derivative thereof. In one embodiment the fragment is capable of performing the above signal peptide activity, or retains the antigenic-binding properties of ANP-SP (1-25), ANP-SP (1-10), or ANP-SP (16-25), or other polypeptide of the invention or polypeptide described herein.

The term “isolated” as applied to the polynucleotide or polypeptide sequences disclosed herein is used to refer to sequences that are removed from their natural cellular environment. An isolated molecule may be obtained by any method or combination of methods including biochemical, recombinant, and synthetic techniques. The polynucleotide or polypeptide sequences may be prepared by at least one purification step.

The term “purified” as used herein does not require absolute purity. Purified refers in one embodiment to at least 90%, or 95%, or 98%, or 99% homogeneity of a polynucleotide, polypeptide antibody, or host cell in a sample. The term should be similarly understood in relation to other molecules and constructs described herein.

The term “isolated” as applied to a cell or host cell describes a cell or host cell that has been obtained or removed from an organism or from its natural environment and is subsequently maintained in a laboratory environment as known in the art. The term is not limited to single cells, per se, but refers to a cell or host cell comprised in a cell culture and can include a single cell or single host cell.

The term “recombinant” refers to a polynucleotide sequence that is removed from sequences that surround it in its natural context and/or is recombined with sequences that are not present in its natural context.

A “recombinant” polypeptide sequence is produced by translation from a “recombinant” polynucleotide sequence.

As used herein, the term “variant” refers to polynucleotide or polypeptide sequences different from the specifically identified sequences, wherein one or more nucleotides or amino acid residues is deleted, substituted, or added. Variants may be naturally occurring allelic variants, or non-naturally occurring variants. Variants may be from the same or from other species and may encompass homologues, paralogues and orthologues. In certain embodiments, variants of the polypeptides useful in the invention have biological activities including signal peptide activity or antigenic binding properties that are the same or similar to those of the parent polypeptides or polynucleotides. The term “variant” with reference to polynucleotides and polypeptides encompasses all forms of polynucleotides and polypeptides as defined herein.

Variant polynucleotide sequences exhibit at least 50%, at least 60%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to a sequence of the present invention. Identity is found over a comparison window of at least 10 nucleotide positions, at least 15 nucleotide positions, at least 20 nucleotide positions, at least 27 nucleotide positions, at least 40 nucleotide positions, at least 50 nucleotide positions, at least 60, or at least 70 nucleotide positions or over the entire length of a polynucleotide of SEQ ID NO:13, SEQ ID NO:15, SEQ ID NO:17 or other polynucleotides disclosed herein.

Polynucleotide sequence identity may be calculated over the entire length of the overlap between a candidate and subject polynucleotide sequences using global sequence alignment programs (e.g. Needleman, S. B. and Wunsch, C. D. (1970) J. Mol. Biol. 48, 443-453). A full implementation of the Needleman-Wunsch global alignment algorithm is found in the needle program in the EMBOSS package (Rice, P. Longden, I. and Bleasby, A. EMBOSS: The European Molecular Biology Open Software Suite, Trends in Genetics June 2000, vol 16, No 6, pp. 276-277) which can be obtained from http://www.hgmp.mrc.ac.uk/Software/EMBOSS/. The European Bioinformatics Institute server also provides the facility to perform EMBOSS-needle global alignments between two sequences on line at http:/www.ebi.ac.uk/emboss/align/.

Alternatively the GAP program may be used which computes an optimal global alignment of two sequences without penalizing terminal gaps. GAP is described in the following paper: Huang, X. (1994) On Global Sequence Alignment (Computer Applications in the Biosciences 10, 227-235).

Polynucleotide variants also encompass those which exhibit a similarity to one or more of the specifically identified sequences that is likely to preserve the functional equivalence of those sequences and which could not reasonably be expected to have occurred by random chance. This program finds regions of similarity between the sequences and for each such region reports an “E value” which is the expected number of times one could expect to see such a match by chance in a database of a fixed reference size containing random sequences. The size of this database is set by default in the bl2seq program. For small E values, much less than one, the E value is approximately the probability of such a random match.

Variant polynucleotide sequences preferably exhibit an E value of less than 1×10⁻⁵, less than 1×10⁻⁶, less than 1×10⁻⁹, less than 1×10⁻¹², less than 1×10⁻¹⁵, less than 1×10⁻¹⁸ or less than 1×10⁻²¹ when compared with any one of the specifically identified sequences.

Polynucleotide sequence identity and similarity can also be determined in the following manner. The subject polynucleotide sequence is compared to a candidate polynucleotide sequence using sequence alignment algorithms and sequence similarity search tools such as in Genbank, EMBL, Swiss-PROT and other databases. Nucleic Acids Res 29:1-10 and 11-16, 2001 provides examples of online resources.

Use of BLASTN is preferred for use in the determination of sequence identity for polynucleotide variants according to the present invention.

BLASTN (from the BLAST suite of programs, version 2.2.18 April 2008 in bl2seq (Tatiana A. et al, FEMS Microbiol Lett. 174:247-250 (1999), Altschul et al., Nuc. Acis Res 25:3389-3402, (1997)), is publicly available from NCBI (ftp://ftp.ncbi.nih.gov/blast/) or from NCB1 at Bethesda, Md., USA. The default parameters of bl2seq are utilized except that filtering of low complexity parts should be turned off.

The identity of polynucleotide sequences may be examined using the following UNIX command line parameters:
bl2seq −i nucleotideseq1 −j nucleotideseq2 −F F −p blastn

The parameter −F F turns off filtering of low complexity sections. The parameter −p selects the appropriate algorithm for the pair of sequences. The bl2seq program reports sequence identity as both the number and percentage of identical nucleotides in a line “Identities=”.

Alternatively, variant polynucleotides are polynucleotides that hybridize to the specified polynucleotide sequence, or a complement thereof under stringent conditions.

The term “hybridize under stringent conditions”, and grammatical equivalents thereof, refers to the ability of a polynucleotide molecule to hybridize to a target polynucleotide molecule (such as a target polynucleotide molecule immobilized on a DNA or RNA blot, such as a Southern blot or Northern blot) under defined conditions of temperature and salt concentration. The ability to hybridize under stringent hybridization conditions can be determined by initially hybridizing under less stringent conditions then increasing the stringency to the desired stringency.

With respect to polynucleotide molecules greater than about 100 bases in length, typical stringent hybridization conditions are no more than 25 to 30° C. (for example, 10° C.) below the melting temperature (Tm) of the native duplex (see generally, Sambrook et al., Eds, 1987, Molecular Cloning, A Laboratory Manual, 2nd Ed. Cold Spring Harbor Press; Ausubel et al., 1987, Current Protocols in Molecular Biology, Greene Publishing, incorporated herein by reference). Tm for polynucleotide molecules greater than about 100 bases can be calculated by the formula Tm=81.5+0.41% (G+C-log (Na+) (Sambrook et al., Eds, 1987, Molecular Cloning, A Laboratory Manual, 2nd Ed, Cold Spring Harbor Press; Bolton and McCarthy, 1962, PNAS 84:1390). Typical stringent conditions for a polynucleotide of greater than 100 bases in length would be hybridization conditions such as prewashing in a solution of 6×SSC, 0.2% SDS; hybridizing at 65° C., 6×SSC, 0.2% SDS overnight; followed by two washes of 30 minutes each in 1×SSC, 0.1% SDS at 65° C. and two washes of 30 minutes each in 0.2×SSC, 0.1% SDS at 65° C.

In one embodiment stringent conditions use 50% formamide, 5×SSC, 50 mM sodium phosphate (pH 6.8), 0.1% sodium pyrophosphate, 5×Denhardt's solution, sonicated salmon sperm DNA (50 μg/ml), OA % SDS, and 10% dextran sulphate at 42° C., with washes at 42° C. in 0.2×SSC and 50% formamide at 55° C., followed by a wash comprising of 0.1×SSC containing EDTA at 55° C.

With respect to polynucleotide molecules having a length less than 100 bases, exemplary stringent hybridization conditions are 5 to 10° C. below Tm. On average, the Tin of a polynucleotide molecule of length less than 100 bp is reduced by approximately (500/oligonucleotide length)° C.

With respect to the DNA mimics known as peptide nucleic acids (PNAs) (Nielsen et al., Science. 1991 Dec. 6; 254(5037):1497-500) Tm values are higher than those for DNA-DNA or DNA-RNA hybrids, and can be calculated using the formula described in Giesen et al., Nucleic Acids Res. 1998 Nov. 1; 26(21):5004-6. Exemplary stringent hybridization conditions for a DNA-PNA hybrid having a length less than 100 bases are 5 to 10° C. below the Tm.

Variant polynucleotides also encompasses polynucleotides that differ from the sequences of the invention but that, as a consequence of the degeneracy of the genetic code, encode a polypeptide having similar activity to a polypeptide encoded by a polynucleotide of the present invention. A sequence alteration that does not change the amino acid sequence of the polypeptide is a “silent variation”. Except for ATG (methionine) and TGG (tryptophan), other codons for the same amino acid may be changed by art recognized techniques, e.g., to optimize codon expression in a particular host organism.

Polynucleotide sequence alterations resulting in conservative substitutions of one or several amino acids in the encoded polypeptide sequence without significantly altering its biological activity are also included in the invention. A skilled artisan will be aware of methods for making phenotypically silent amino acid substitutions (see, e.g., Bowie et al., 1990, Science 247, 1306⁹).

Variant polynucleotides due to silent variations and conservative substitutions in the encoded polypeptide sequence may be determined using the bl2seq program via the tblastx algorithm as described above.

The term “variant” with reference to polypeptides also encompasses naturally occurring, recombinantly and synthetically produced polypeptides. Variant polypeptide sequences preferably exhibit at least 50%, at least 60%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to a sequence of the present invention. Identity is found over a comparison window of at least 5, at least 7, at least 10, at least 15, at least 20, or at least 24 amino acid positions, or over the entire length of a polypeptide of SEQ ID NO:12, SEQ ID NO: 14 or SEQ ID NO:16 or other polypeptides disclosed or used in the invention.

Polypeptide variants also encompass those which exhibit a similarity to one or more of the specifically identified sequences that is likely to preserve the functional equivalence of those sequences and which could not reasonably be expected to have occurred by random chance. As discussed above, in the case of ANP-SP variants function may be as either a signal polypeptide, or antigenic polypeptide, or both.

Polypeptide sequence identity and similarity can be determined in the following manner. The subject polypeptide sequence is compared to a candidate polypeptide sequence using BLASTP (from the BLAST suite of programs, version 2.2.18 [April 2008]) in bl2seq, which is publicly available from NCBI (ftp://ftp.ncbi.nih.gov/blast/). The default parameters of bl2seq are utilized except that filtering of low complexity regions should be turned off.

The similarity of polypeptide sequences may be examined using the following UNIX command line parameters:
bl2seq −i peptideseq1 −j peptideseq2 −F F −p blastp

The parameter −F F turns off filtering of low complexity sections. The parameter −p selects the appropriate algorithm for the pair of sequences. This program finds regions of similarity between the sequences and for each such region reports an “E value” which is the expected number of times one could expect to see such a match by chance in a database of a fixed reference size containing random sequences. For small E values, much less than one, this is approximately the probability of such a random match.

Variant polypeptide sequences commonly exhibit an E value of less than 1×10⁻⁵, less than 1×10⁶, less than 1×10⁻⁹, less than 1×10⁻¹², less than 1×10⁻¹⁵, less than 1×10⁻¹⁸ or less than 1×10⁻²¹ when compared with any one of the specifically identified sequences.

Polypeptide sequence identity may also be calculated over the entire length of the overlap between a candidate and subject polypeptide sequences using global sequence alignment programs. EMBOSS-needle (available at http:/www.ebi.ac.uk/emboss/align/) and GAP (Huang, X. (1994) On Global Sequence Alignment. Computer Applications in the Biosciences 10, 227-235.) as discussed above are also suitable global sequence alignment programs for calculating polypeptide sequence identity.

Use of BLASTP as described above is preferred for use in the determination of polypeptide variants according to the present invention.

In one embodiment variants include peptides who's sequence differs from the human ANP-SP (1-25) SEQ ID NO:14, ANP-SP (16-25) SEQ ID NO:12 or ANP-SP (1-10) SEQ ID NO:16 herein by one or more conservative amino acid substitutions, deletions, additions or insertions which do not affect the biological activity of the peptide. Conservative substitutions typically include the substitution of one amino acid for another with similar characteristics, e.g., substitutions within the following groups: valine, glycine; glycine, alanine; valine, isoleucine, leucine; aspartic acid, glutamic acid; asparagines, glutamine; serine, threonine; lysine, arginine; and phenylalanine, tyrosine. Examples of conservative substitutions can also be found in the sequences of ANP-SP in FIGS. 2A and 2B whereby the substitutions in different mammalian species compared to the human sequence are shown. Examples of conservative and semi-conservative substitutions can be taken from Table 1 below.

TABLE 1

| | Exemplary | Preferred |
|---|---|---|
| Original Residue | Substitutions | Substitution |
| Ala (A) | val; leu; ile; pro | val |
| Arg (R) | lys; gln; asn | lys |
| Asn (N) | gln; his; lys; arg | gln |
| Asp (D) | glu | glu |
| Cys (C) | ser | ser |
| Gln (Q) | asn; his | his |
| Glu (E) | asp | asp |
| Gly (G) | pro; ala | ala |
| His (H) | asn; gln; lys; arg | arg |
| Ile (I) | leu; val; met; ala; | leu |
| | phe; norleucine | |
| Leu (L) | norleucine; ile; | phe; val |
| | val; met; ala; phe | |
| Lys (K) | arg; gln; asn | arg |
| Met (M) | leu; phe; ile | leu |
| Phe (F) | leu; val; ile; ala; | val |
| | tyr | |
| Pro (P) | ala | ala |
| Ser (S) | Thr; gly | gly |
| Thr (T) | Ser; ala; pro | Ser; ala |
| Trp (W) | tyr; phe | Tyr |
| Tyr (Y) | trp; phe; thr; ser | phe |
| Val (V) | ile; leu; met; phe; | leu |
| | ala; norleucine | |

Naturally occurring residues are divided into groups based on common side-chain properties:
(1) hydrophobic: norleucine, met, ala, val, leu, ile;
(2) neutral hydrophilic: cys, ser, thr;
(3) acidic: asp, glu;
(4) basic: asn, gin, his, lys, arg:
(5) residues that influence chain orientation: gly, pro; and
(6) aromatic: trp, tyr, phe.

Non-conservative substitutions will entail exchanging a member of one of these classes for a member of another class. See for example S being substituted with M at ANP-SP 2.

Other variants include peptides with modifications which influence peptide stability. Such analogs may contain, for example, one or more non-peptide bonds (which replace the peptide bonds) in the peptide sequence. Also included are analogs that include residues other than naturally occurring L-amino acids, e.g. D-amino acids or non-naturally occurring synthetic amino acids, e.g. beta or gamma amino acids and cyclic analogs.

Substitutions, deletions, additions or insertions may be made by mutagenesis methods known in the art. A skilled worker will be aware of methods for making phenotypically silent amino acid substitutions. See for example Bowie et al., 1990, Science 247, 1306.⁹, Kunkel, T; 1985, PNAS, 82 p 488.²⁷

Also included within the polypeptides of the invention are those which have been modified during or after synthesis for example by biotinylation, benzylation, glycosylation, phosphorylation, amidation, by derivatization using blocking/protecting groups and the like. Such modifications may increase stability or activity of the polypeptide. Such modifications are well known in the art. See for example, Sambrook and Ausubel (supra), and Lundblad, R, CRC Press, 1995.²⁸

The term “genetic construct” refers to a polynucleotide molecule, usually double-stranded DNA, which may have inserted into it another polynucleotide molecule (the insert polynucleotide molecule) such as, but not limited to, a cDNA molecule. A genetic construct may contain the necessary elements that permit transcribing the insert polynucleotide molecule, and, optionally, translating the transcript into a polypeptide. The insert polynucleotide molecule may be derived from the host cell, or may be derived from a different cell or organism and/or may be a recombinant polynucleotide. Once inside the host cell the genetic construct may become integrated in the host chromosomal DNA. The genetic construct may be linked to a vector.

The term “vector” refers to a polynucleotide molecule, usually double stranded DNA, which is used to transport the genetic construct into a host cell. The vector may be capable of replication in at least one additional host system, such as E. coli.

The term “expression construct” refers to a genetic construct that includes the necessary elements that permit transcribing the insert polynucleotide molecule, and, optionally, translating the transcript into a polypeptide. An expression construct typically comprises in a 5′ to 3′ direction:
(a) a promoter functional in the host cell into which the construct will be transformed,
(b) the polynucleotide to be expressed, and
(c) a terminator functional in the host cell into which the construct will be transformed.

The term “coding region” or “open reading frame” (ORF) refers to the sense strand of a genomic DNA sequence or a cDNA sequence that is capable of producing a transcription product and/or a polypeptide under the control of appropriate regulatory sequences. The coding sequence is identified by the presence of a 5′ translation start codon and a 3′ translation stop codon. When inserted into a genetic construct, a “coding sequence” is capable of being expressed when it is operably linked to promoter and terminator sequences and/or other regulatory elements.

“Regulatory elements” and “polynucleotide regulatory elements” mean any element that controls or influences the expression of a polynucleotide insert from a vector, genetic construct or expression cassette and includes promoters, transcription control sequences, translation control sequences, origins of replication, tissue-specific regulatory elements, temporal regulatory elements, enhancers, polyadenylation signals, repressors and terminators. Regulatory elements can be homologous or heterologous to the polynucleotide insert to be expressed from a vector, genetic construct or expression cassette according to the invention.

“Homologous” as used herein with reference to the relationship between a polynucleotide regulatory element (PRE) and the sequence to which the PRE is operably linked in a genetic construct means that the PRE is normally associated in nature with the coding sequence to which it is operably linked in the construct. A homologous polynucleotide regulatory element may be operably linked to a polynucleotide of interest such that the polynucleotide of interest can be expressed from a, vector, genetic construct or expression cassette according to the invention.

“Heterologous” as used herein with reference to the relationship between a polynucleotide regulatory element (PRE) and the sequence to which the PRE is operably linked in a genetic construct means that the PRE is not normally associated in nature with the coding sequence to which it is operably linked in the construct. Such PREs may include promoters normally associated with different genes (other than ANP), and/or promoters isolated from any other bacterial, viral, eukaryotic, or mammalian cell.

“Operably-linked” means that the sequence to be expressed is placed under the control of regulatory elements that include promoters, transcription control sequences, translation control sequences, origins of replication, tissue-specific regulatory elements, temporal regulatory elements, enhancers, polyadenylation signals, repressors and terminators.

The term “noncoding region” refers to untranslated sequences that are upstream of the translational start site and downstream of the translational stop site. These sequences are also referred to respectively as the 5′ UTR and the 3′ UTR. These regions include elements required for transcription initiation and termination and for regulation of translation efficiency.

Terminators are sequences, which terminate transcription, and are found in the 3′ untranslated ends of genes downstream of the translated sequence. Terminators are important determinants of mRNA stability and in some cases have been found to have spatial regulatory functions.

The term “promoter” refers to nontranscribed cis-regulatory elements upstream of the coding region that regulate gene transcription. Promoters comprise cis-initiator elements which specify the transcription initiation site and conserved boxes such as the TATA box, and motifs that are bound by transcription factors.

The terms “to alter expression of” and “altered expression” of a polynucleotide or polypeptide of the invention, are intended to encompass the situation where genomic DNA corresponding to a polynucleotide of the invention is modified thus leading to altered expression of a polynucleotide or polypeptide of the invention. Modification of the genomic DNA may be through genetic transformation or other methods known in the art for inducing mutations. The “altered expression” can be related to an increase or decrease in the amount of messenger RNA and/or polypeptide produced and may also result in altered activity of a polypeptide due to alterations in the sequence of a polynucleotide and polypeptide produced.

“Subject” as used herein is preferably a mammal and includes human, and non-human mammals such as cats, dogs, horses, cows, sheep, deer, mice, rats, primates (including gorillas, rhesus monkeys and chimpanzees), possums and other domestic farm or zoo animals. Preferably, the mammal is human.

The term “presentation” as used herein refers to presentation of a subject at a medical facility such as a clinic or hospital.

A “therapeutically effective amount” or “therapeutically effective dose” as used herein means an amount sufficient to produce the desired physiological effect or an amount capable of achieving the desired result, particularly for treating the desired disease or condition, including reducing or eliminating one or more symptoms or manifestations of the disease or condition.

The term “treat”, “treating” or “treatment” and “preventing” refer to therapeutic or prophylactic measures which alleviate, ameliorate, manage, prevent, restrain, stop or reverse progression of ACD, or cardiac transplant rejection or effects thereof, particularly of ACS. The subject may show observable or measurable (statistically significant) reduction in one or more of TnI, TnT BNP, N-BNP, and other usual clinical markers known to those skilled in the art, indicating improvement.

The term “mass spectrometry” as used herein refers to methods of filtering, detecting, and measuring ions based on their mass to charge ratio. See for example U.S. Pat. No. 5,719,060, U.S. Pat. No. 6,204,500, U.S. Pat. No. 6,107,623, U.S. Pat. No. 6,124,137, U.S. Pat. No. 6,225,047, U.S. Pat. No. 6,268,144, U.S. Pat. No. 7,057,165, and U.S. Pat. No. 7,045,366. Common mass spectrometry techniques include matrix-assisted laser desorption ionization (MALDI) and surface-enhanced laser desorption ionization (SELDI). Both may be coupled with time of flight analysers (MALDI-TOF and SELDI-TOF) which allow for analysis of analytes at femtomole levels in very short ion pulses.

Versions of SELDI discussed for example in U.S. Pat. No. 5,719,600, U.S. Pat. No. 6,124,137, and U.S. Pat. No. 6,225,047 which are useful in this invention include Surface-Enhanced Affinity Capture (SEAC), Surface-Enhanced Neat Desorption (SEND), and Surface-Enhanced Photolabile Attachment and Release (SEPAR).

It is intended that reference to a range of numbers disclosed herein (for example 1 to 10) also incorporates reference to all related numbers within that range (for example, 1, 1.1, 2, 3, 3.9, 4, 5, 6, 6.5, 7, 8, 9 and 10) and also any range of rational numbers within that range (for example 2 to 8, 1.5 to 5.5 and 3.1 to 4.7) and, therefore, all sub-ranges of all ranges expressly disclosed herein are expressly disclosed. These are only examples of what is specifically intended and all possible combinations of numerical values between the lowest value and the highest value enumerated are to be considered to be expressly stated in this application in a similar manner.

### DETAILED DESCRIPTION OF THE INVENTION

Human atrial natriuretic peptide (ANP) is a member of the cardiac natriuretic peptide family. As shown in FIG. 1, preproANP is a 151 amino acid molecule. The signal peptide ANP-SP (1-25) (shown in bold in FIG. 2B) is cleaved to give preproANP (26-151). PreproANP (26-151) is in turn further processed to give bioactive forms preproANP (124-151) and prepro ANP (26-123). PreproANP is a 28 amino acid peptide that is synthesised, stored, and released by atrial myocytes. The mature human ANP is shown underlined in FIG. 2B. It is likely that ANP-SP is degraded into smaller fragments by signal peptidase (SPP); usually near the hydrophobic central region of the ANP-SP (1-25) sequence.

It has long been thought that the functional role of the ANP-SP is limited to controlling the trafficking of ANP in the endoplasmic reticulum. Once this is achieved it has been assumed that the signal peptide is then degraded without ever being secreted from the cell.

Very recently, it has been postulated (but not proven) that ANP-SP may appear in the circulation (WO 2005/052593; US 2005/0244904). Based on this postulation ANP-SP has been suggested for use as a circulating biomarker for cardiac disease. The present applicants have made a further and highly unexpected finding. In patients with acute myocardial infarction (AMI) the circulating concentration of ANP-SP is highest in the first few hours following the onset of the patient's symptoms—in fact, at the time of presentation to the hospital or clinic. This is contrary to expectations that ANP-SP levels would be correlated with N-ANP levels and could therefore be expected to reach their peak 12 to 24 hours from onset of, or clinical presentation with ACD, cardiac transplant rejection, or with an undiagnosed or suspected ACD or pulmonary disorder. Levels observed in the first few hours are surprisingly very high often reaching a peak some five to fifteen, commonly three to seven fold higher than levels in a normal control population.

The level of ANP-SP remains higher than ANP-SP levels in a control population. These findings suggest ANP-SP is useful as a very clear early stage marker of cardiac transplant rejection, ACD including acute coronary syndromes (ACS) such as AMI, particularly non-ST elevated MI, acute cardiac ischemia and may be used to distinguish ACD from pulmonary disorders.

Based on these surprising findings, the applicants have determined for the first time, that it would be useful to screen for circulating ANP-SP or variants or fragments thereof, as well as, or alternately nucleotide sequences encoding ANP-SP or the variants and fragments thereof in a biological sample taken from a subject within four or two hours of onset of, or at clinical presentation with the disorder.

Useful in the invention are antigenic fragments or variants of ANP-SP which are least 4 or 5 amino acids in length. Peptides having as few as 4 amino acids are known to be biologically active. See for example Gilchrist et al, Biology and Reproduction, 21, 732-739, 2004; and Sela et al., Behring Ins. Mitt., 91, 54-66, 1992. Particularly useful fragments are at the N-terminus (1-13) or C-terminus (16-25) of ANP-SP. Examples of specific antigenic peptides are ANP-SP (16-25) (SEQ ID NO:12) and ANP-SP (1-10) (SEQ ID NO:16). The corresponding nucleotide sequences are given in SEQ ID NOs: 13 and 17 respectively. These sequences are provided by the applicants for the first time. Both the nucleic acid molecules and peptides form aspects of the invention.

Accordingly, in a first aspect, the invention provides an isolated nucleic acid molecule encoding an ANP-SP (16-25) (SEQ ID NO:12) or ANP-SP (1-10) (SEQ ID NO:16) or a variant or fragment thereof wherein said nucleic acid is
(a) SEQ ID NO:13 or SEQ ID NO:17 or a variant or fragment thereof;

(b) a sequence which has 70%, 75%, 80%, 90%, 95% or 99% sequence identity to SEQ ID NO:13 or SEQ ID NO:17;
(c) a sequence which hybridises under stringent conditions to SEQ ID NO:13 or SEQ ID NO:17 or a fragment or variant thereof;
(d) a sequence of at least 10 nucleotides in length, capable of hybridising to the sequence of any one of (a) to (c) under stringent conditions;
(e) a complement of any one of (a) to (d); with the proviso that the sequence is not SEQ ID NO: 15.

The invention also provides isolated ANP-SP polypeptides encoded by a nucleic acid molecule of the invention.

Specific polypeptides of the invention include polypeptides having the amino acid sequence of SEQ ID NO: 12 or SEQ ID NO:16 as set forth in the accompanying sequence listing. Also contemplated as part of the invention are variants and fragments of these polypeptides as defined herein, or amino acid sequences having at least 70%, 75%, 80%, 85%, 90%, 95% or 99% amino acid identity to the polypeptide of SEQ ID NO:12 or SEQ ID NO:16. In one embodiment the variants or fragments are functionally equivalent variants or fragments. That is the variants or fragments maintain the function of SEQ ID NO:12 or SEQ ID NO:16 as antigens or signal peptides.

The nucleic acid molecules of the invention or otherwise described herein are in one embodiment isolated. They can be isolated from a biological sample using a variety of techniques known to those of ordinary skill in the art. By way of example, such polynucleotides can be isolated through use of the polymerase chain reaction (PCR) described in Mullis et al., Eds. 1994 The Polymerase Chain Reaction, Birkhauser. The nucleic acid molecules of the invention can be amplified using primers, as defined herein, derived from the polynucleotide sequences of the invention. (See for example Mullis, Sambrook supra; and Molecular Diagnostic PCR Handbook Gerrit, V et al., Springer, 2005).

Further methods for isolating polynucleotides include use of all, or portions of, the polynucleotide of the invention, particularly a polynucleotide having the sequence set forth in SEQ ID NO:13 or SEQ ID NO:17 as hybridization probes. The technique of hybridizing labeled polynucleotide probes to polynucleotides immobilized on solid supports such as nitrocellulose filters or nylon membranes, can be used to screen genomic or cDNA libraries. Similarly, probes may be coupled to beads and hybridized to the target sequence. Isolation can be effected using known art protocols such as magnetic separation. Exemplary stringent hybridization and wash conditions are as given above.

Polynucleotide fragments may be produced by techniques well-known in the art such as restriction endonuclease digestion and oligonucleotide synthesis.

A partial polynucleotide sequence may be used as a probe, in methods well-known in the art to identify the corresponding full length polynucleotide sequence in a sample. Such methods include PCR-based methods, 5′RACE (Methods Enzymol. 218: 340-56 (1993); Sambrook et al., Supra) and hybridization-based method, computer/database-based methods. Detectable labels such as radioisotopes, fluorescent, chemiluminescent and bioluminescent labels may be used to facilitate detection. Inverse PCR also permits acquisition of unknown sequences, flanking the polynucleotide sequences disclosed herein, starting with primers based on a known region (Triglia et al., Nucleic Acids Res 16, 8186, (1998)) The method uses several restriction enzymes to generate a suitable fragment in the known region of a gene. The fragment is then circularized by intramolecular ligation and used as a PCR template. Divergent primers are designed from the known region. In order to physically assemble full-length clones, standard molecular biology approaches can be utilized (Sambrook et al., Supra). Primers and primer pairs which allow amplification of polynucleotides of the invention, also form a further aspect of this invention.

Variants (including orthologues) may be identified by the methods described. Variant polynucleotides may be identified using PCR-based methods (Mullis et al., Eds. 1994 The Polymerase Chain Reaction, Birkhauser). Typically, the polynucleotide sequence of a primer, useful to amplify variants of polynucleotide molecules by PCR, may be based on a sequence encoding a conserved region of the corresponding amino acid sequence.

Further methods for identifying variant polynucleotides include use of all, or portions of, the specified polynucleotides as hybridization probes to screen genomic or cDNA libraries as described above. Typically probes based on a sequence encoding a conserved region of the corresponding amino acid sequence may be used. Hybridisation conditions may also be less stringent than those used when screening for sequences identical to the probe.

The variant sequences, including both polynucleotide and polypeptide variants, may also be identified by the computer-based methods discussed above. In addition, multiple sequence alignments of a group of related sequences can be carried out with CLUSTALW (Thompson, et al., Nucleic Acids Research, 22:4673-4680 (1994), http://www-igbmc.ustrasbg.fr/BioInfo/ClustalW/Top.html) or T-COFFEE (Cedric Notredame et al., J. Mol. Biol. 302: 205-217 (2000))) or PILEUP, which uses progressive, pairwise alignments. (Feng et al., J. Mol. Evol. 25, 351 (1987)).

Pattern recognition software applications are available for finding motifs or signature sequences. For example, MEME (Multiple Em for Motif Elicitation) finds motifs and signature sequences in a set of sequences, and MAST (Motif Alignment and Search Tool) uses these motifs to identify similar or the same motifs in query sequences. The MAST results are provided as a series of alignments with appropriate statistical data and a visual overview of the motifs found. MEME and MAST were developed at the University of California, San Diego.

PROSITE (Bairoch et al., Nucleic Acids Res. 22, 3583 (1994); Hofmann et al., Nucleic Acids Res. 27, 215 (1999)) is a method of identifying the functions of uncharacterized proteins translated from genomic or cDNA sequences. The PROSITE database (www.expasy.org/prosite) contains biologically significant patterns and profiles and is designed so that it can be used with appropriate computational tools to assign a new sequence to a known family of proteins or to determine which known domain(s) are present in the sequence (Falquet et al., Nucleic Acids Res. 30, 235 (2002)). Prosearch is a tool that can search SWISS-PROT and EMBL databases with a given sequence pattern or signature.

Proteins can be classified according to their sequence relatedness to other proteins in the same genome (paralogues) or a different genome (orthologues). Orthologous genes are genes that evolved by speciation from a common ancestral gene and normally retain the same function as they evolve. Paralogous genes are genes that are duplicated within a genome and genes may acquire new specificities or modified functions which may be related to the original one. Phylogenetic analysis methods are reviewed in Tatusov et al., Science 278, 631-637, 1997.

As noted above, the invention also relates to ANP-SP polypeptides encoded by the nucleic acid molecules of the invention, and includes variants and fragments of these polypeptides.

In addition to the computer/database methods described above, polypeptide variants may be identified by physical methods, for example by screening expression libraries using antibodies raised against polypeptides of the invention (Sambrook et al., Molecular Cloning: A Laboratory Manual, 2nd Ed. Cold Spring Harbor Press, 1987) by recombinant DNA techniques also described by Sambrook et al. or by identifying polypeptides from natural sources with the aid of such antibodies.

Polypeptides, including variant polypeptides, may be prepared using peptide synthesis methods well known in the art such as direct peptide synthesis using solid phase techniques (e.g. Merrifield, 1963, in J. Am. Chem. Soc. 85, 2149; Stewart et al., 1969, in Solid-Phase Peptide Synthesis, WH Freeman Co, San Francisco Calif.; Matteucci et al. J. Am. Chem. Soc. 103:3185-3191, 1981, and Atherton et al., in Solid Phase Peptide Synthesis: a practical approach, IRL press (1989)) or automated synthesis, for example using a Synthesiser from Applied Biosystems (California, USA). Mutated forms of the polypeptides may also be produced using synthetic methods such as site-specific mutagensis of the DNA encoding the amino acid sequence as described by Adelmen et al; DNA 2, 183 (1983). See also Protein Protocols Handbook; Walker, J. Humana Press 2002.

The polypeptides and variant polypeptides herein are in one embodiment isolated. They may be isolated or purified from natural sources using a variety of techniques that are well known in the art (e.g. Deutscher, 1990, Ed, Methods in Enzymology, Vol. 182, Guide to Protein Purification, and Protein Protocols Handbook, supra). Technologies include HPLC, ion-exchange chromatography, and immunochromatography but are not limited thereto.

Alternatively the polypeptides and variant polypeptides may be expressed recombinantly in suitable host cells and separated from the cells as discussed below. The polypeptides and variants have utility in generating antibodies, and generating ligands amongst other uses.

The genetic constructs described herein may comprise one or more of the disclosed polynucleotide sequences and/or polynucleotides encoding the disclosed polypeptides, of the invention and may be useful for transforming, for example, bacterial, fungal, insect, mammalian or plant organisms. The genetic constructs of the invention are intended to include expression constructs as herein defined. Included are vectors (such as pBR322, pUC18, pU19, Mp18, Mp19, ColE1, PCR1 and pKRC), phages (such as lambda gt10), and M13 plasmids (such as pBR322, pACYC184, pT127, RP4, p1J101, SV40 and BPV), cosmids, YACS, BACs shuttle vectors such as pSA3, PAT28 transposons (such as described in U.S. Pat. No. 5,792,294) and the like.

The constructs may conveniently include a selection gene or selectable marker. Typically an antibiotic resistance marker such as ampicillin, methotrexate, or tetracycline is used.

Promoters useful in the constructs include β-lactamase, alkaline phosphatase, tryptophan, and tac promoter systems which are all well known in the art. Yeast promoters include 3-phosphoglycerate kinase, enolase, hexokinase, pyruvate decarboxylase, glucokinase, and glyceraldehydrate-3-phosphanate dehydrogenase but are not limited thereto.

Enhancers may also be employed to act on the promoters to enhance transcription. Suitable enhancers for use herein include SV40 enhancer, cytomeglovirus early promoter enhancer, globin, albumin, insulin and the like.

Methods for producing and using genetic constructs and vectors are well known in the art and are described generally in Sambrook et al., (supra), and Ausubel et al., Current Protocols in Molecular Biology, Greene Publishing, 1987. Methods for transforming selected host cells with the vectors are also known, for example, the calcium chloride treatment described by Cohen, S N; PNAS 69, 2110, 1972.

For a general discussion of constructs, promoters, enhancers, and host cells, see Principles of Gene Manipulation and Genomics; Primrose, S et al., Blackwell Publishing 2006, Ed. 7., and From Genes to Genomes: Concepts and Applications of DNA Technology, Dale, J et al., Wiley-Interscience, 2007, Ed. 2.

Host cells comprising the genetic constructs and vectors described may be derived from prokaryotic or eukaryotic sources, for example yeast, bacteria, fungi, insect (eg baculovirus), animal, mammalian or plant organisms. In one embodiment the host cells are isolated host cells. Prokaryotes most commonly employed as host cells are strains of E. coli. Other prokaryotic hosts include Pseudomonas, Bacillus, Serratia, Klebsiella, Streptomyces, Listeria, Saccharomyces, Salmonella and Mycobacteria but are not limited thereto.

Eukaryotic cells for expression of recombinant protein include but are not limited to Vero cells, HeLa, CHO (Chinese Hamster ovary cells), 293, BHK cells, MDCK cells, and COS cells as well as prostate cancer cell lines such as PrEC, LNCaP, Du 145 and RWPE-2. The cells are available from ATCC, Virginia, USA.

Prokaryotic promoters compatible with expression of nucleic acid molecules of the invention include known art constitutive promoters (such as the int promoter of bacteriophage lamda and the bla promoter of the beta-lactamase gene sequence of pBR322) and regulatable promoters (such as lacZ, recA and gal). A ribosome binding site upstream of the coding sequence may also be required for expression.

Host cells comprising genetic constructs, such as expression constructs, are useful in methods for recombinant production of polypeptides. Such methods are well known in the art (see for example Sambrook et al. supra). The methods commonly involve the culture of host cells in an appropriate medium in conditions suitable for or conducive to, expression and selection of a polypeptide of the invention. Cells with a selectable marker may additionally be grown on medium appropriate for selection of host cells expressing a polypeptide of the invention. Transformed host cells expressing a polypeptide of the invention are selected and cultured under conditions suitable for expression of the polypeptide. The expressed recombinant polypeptide, may be separated and purified from the culture medium using methods well known in the art including ammonium sulfate precipitation, ion exchange chromatography, gel filtration, affinity chromatography, electrophoresis and the like (e.g. Deutscher, Ed, 1990, Methods in Enzymology, Vol 182, Guide to Protein Purification). Host cells may also be useful in methods for production of a product generated by an expressed polypeptide of the invention.

In another aspect, the present invention provides a method for predicting, diagnosing or monitoring an acute cardiac disorder (ACD) in a subject, the method comprising:

measuring the level of ANP-SP in a biological sample obtained from the subject within four or two hours of onset of the ACD, or within four or two hours of presentation with ACD; and comparing the level of said ANP-SP with the ANP-SP level from a control wherein a measured level of ANP-SP higher than the control level is indicative of ACD.

In another aspect the invention provides a method for monitoring a response to treatment of a an acute cardiac disorder (ACD) in a subject, the method comprising measuring the level of ANP-SP in a biological sample obtained from the subject within four or two hours of onset of the ACD, or within four or two hours of presentation with the ACD; and comparing the level of said ANP-SP with the ANP-SP level from a control, wherein a change in the measured level of ANP-SP from the control level is indicative of a response to the treatment.

It is known in the art that other cardiac natriuretic peptides such as BNP precursors e.g. BNP 27-102, can be used in predicting or diagnosing a cardiac transplant rejection episode and to distinguish between pulmonary and cardiovascular causes of dyspnea (shortness of breath). See US 2005/0244902. Accordingly, it is similarly predictable that ANP-SP can be used as an early marker of cardiac transplant rejection based on cardiac tissue analysis, and to distinguish pulmonary from acute cardiac disorders.

Accordingly, the invention also provides a method for predicting, diagnosing or monitoring a cardiac transplant rejection episode in a subject, the method comprising measuring the level of ANP-SP in a biological sample obtained from a subject within four hours of heart transplant and comparing the level of said ANP-SP with the ANP-SP level from a control, wherein a measured level of ANP-SP higher than the control level is indicative of transplant rejection.

The invention also provides a method of distinguishing between a pulmonary disorder and an acute cardiac disorder (ACD) in a subject, the method comprising measuring the level of ANP-SP in a biological sample obtained from a subject within four hours of presentation with the disorder; and comparing the level of said ANP-SP with the ANP-SP level from a control wherein a measured ANP-SP level higher than the control level is indicative of ACD.

In one embodiment, the invention provides a method for predicting, diagnosing or monitoring an acute cardiac disorder (ACD), cardiac transplant rejection, or ACD/pulmonary disorder in a subject, the method comprising measuring the level of ANP-SP in a biological sample obtained from the subject within the first four hours of onset of, or clinical presentation with ACD, transplant rejection or ACD/pulmonary disorder. A measured level of ANP-SP is compared with the ANP-SP level from a control wherein a measured level of ANP-SP higher than the control level is indicative of ACD or transplant rejection.

The skilled reader will appreciate that for evaluation purposes, the ANP-SP level requires correlation with a reference value or control value.

As used herein a control can be an individual or group from which ANP-SP samples are taken and a mean ANP-SP level determined. Usually, the individual or group will comprise normal healthy individuals or a group of individuals not known to be suffering from ACD, cardiac transplant rejection or ACD/pulmonary disorder. ANP-SP levels in most individuals are between 14-26 pmol/L, and the mean control level is about 20.8 pmol/L. Alternatively, the control level may be assessed based on a plurality of readings from previously tested individuals or groups. Another example of a control level is a ratiometric measure between ANP-SP and ANP levels in cardiac tissue. The subject's ANP-SP level can be compared to the mean ANP-SP level for that control population. The ANP-SP level in the cardiac tissue control population may be in the order of 1.5 to 3, commonly 2 to 3 or 2.5 to 3 times higher than ANP-SP levels in the normal control population.

Alternatively, the control may be one or more readings or the mean of such readings taken from the same subject at an earlier time. Ascertaining appropriate controls and control levels for particular methods is well known in the art.

It will be appreciated that the step of measuring ANP-SP levels in a sample may be a single measurement on a single sample, or repeated measurements on a number of samples. Accordingly, in one embodiment measurement may comprise 1 to 20 measurements of ANP-SP, 1 to 10, 1 to 5, 1 to 3, 1 or 2, or 2 or 3 measurements, in samples taken at different times within the first four hours, two hours, or within one hour of, onset of or clinical presentation. Single, or repeated measurements outside the four or two hour period may also be taken to establish whether the ANP-SP level has fallen to the normal control level, or cardiac tissue control level.

In one embodiment, the method comprises measuring ANP-SP levels in 1 or 2 samples taken within the first hour of onset or presentation, followed by measuring ANP-SP levels in 1 or 2 samples taken within two to four hours, or two to three hours of onset or presentation, or initial measurement of the ANP-SP level.

As noted above, ANP-SP levels measured within the first four, or two hours of onset or presentation are usually three to seven or five to seven times higher than ANP-SP levels measured in a normal control. As stated above, also included within the ranges are the specific ranges 3 to 6, 3 to 5, 3 to 4, 4 to 7, 4 to 6, 4 to 5, and 5 to 6 times higher.

In another embodiment, a level of ANP-SP in the sample in the range 40 to 300 pmol/L, or 42 to 200 pmol/L, or 45 to 200 pmol/L, or 45 to 150 pmol/L is indicative of ACD, cardiac transplant rejection, or distinguishes ACD from a pulmonary disorder.

As stated above, the ranges also include any values within the range such as 50 to 130 pmol/L, 55 to 120 pmol/L, 60 to 90 pmol/L, and the like.

The biological sample as defined above can be any biological material in which ANP-SP can be located or secreted. In one embodiment the biological sample is a circulatory biological sample, for example blood, serum or plasma. In one embodiment, the biological sample is cardiac tissue.

### Nucleic Acid Assays

The presence of ANP-SP and its level of expression in the sample may be determined according to methods known in the art such as Southern Blotting, Northern Blotting, FISH or quantative PCR to quantitate the transcription of mRNA [(Thomas, Proc. Nat, Acad. Sci. USA 77: 5201-5205 1980), (Jain K K, Med Device Technol. 2004 May; 15(4):14-7)], dot blotting, DNA analysis or in situ hybridization using an appropriately labelled probe, based on the sequences provided herein.

Accordingly, the invention also provides an assay for detecting the presence of a nucleic acid molecule of the invention, in a sample, the method comprising:
(a) contacting the sample with a polynucleotide probe which hybridises to the nucleic acid sequence under stringent hybridisation conditions; and
(b) detecting the presence of a hybridisation complex in the sample.

In one embodiment the nucleic acid molecule is SEQ ID NO:13 or SEQ ID NO:17 or a variant or fragment thereof.

Preferably the hybridisation probe is a labelled probe. Examples of labels include fluorescent, chemiluminescent, radioenzyme and biotin-avidin labels. Labelling and visualisation of labelled probes is carried out according to known art methods such as those above.

For convenience the nucleic acid probe may be immobilized on a solid support including resins (such as polyacrylamides), carbohydrates (such as sepharose), plastics (such as polycarbonate), and latex beads.

As discussed above the nucleic acid molecule probe may preferably be an RNA, cDNA or DNA molecule. In one embodiment the probe is, or includes SEQ ID NO: 13 or SEQ ID NO:17.

Stringent hybridisation conditions are as discussed above.

The expression level of the nucleic acid marker may be determined using known art techniques such as RT-PCR and electrophoresis techniques including SDS-PAGE. Using these techniques the DNA or cDNA sequence of a nucleic acid molecule of the invention, in a subject sample is amplified, and the level of DNA or cDNA or RNA measured.

In an alternate method the DNA, cDNA or RNA level may be measured directly in the sample without amplification.

A currently preferred method is Northern blot hybridization analysis. Probes for use in Northern blot hybridization analysis may be prepared based on the marker sequences identified herein. In one embodiment, a probe includes at least 10, at least 12, at least 15, at least 18, at least 24, at least 30, at least 36, at least 42, at least 51, at least 60, or at least 70 or more contiguous nucleotides of a reference sequence.

Alternatively, the expression level may be measured using reverse transcription based PCR(RT-PCR) assays using primers specific for the nucleic acid sequences. If desired, comparison of the level of the marker in the sample can be made with reference to a control nucleic acid molecule the expression of which is independent of the parameter or condition being measured. A control nucleic acid molecule refers to a molecule in which the level does not differ between the disorder or transplant rejection state and the healthy state. Levels of the control molecule can be used to normalise levels in the compared populations. An example of such a control molecule is GAP-DR The markers of the invention will change levels with the disorder.

### Peptide Assays

In one embodiment the measuring step comprises detecting binding between ANP-SP and a binding agent that binds, (including selectively or specifically binds) ANP-SP or a fragment or variant thereof. As a pre-step in the measurement ANP-SP may be bound with a binding agent that binds ANP-SP or a fragment or variant thereof.

Accordingly, in one embodiment the invention provides an assay for ANP-SP in a biological sample obtained from a subject within four hours from onset of ACD, cardiac transplant rejection, or ACD/pulmonary disorder or within four hours of clinical presentation with ACD, cardiac transplant rejection, or ACD/pulmonary disorder, the assay comprising detecting and measuring the level of ANP-SP in the sample using any known methods.

In another embodiment, the invention provides an assay for ANP-SP comprising:
(a) binding one or more ANP-SP polypeptides from a biological sample, wherein the ANP-SP polypeptide is selected from the group ANP-SP 1-10, and ANP-SP 16-25, or a variant or fragment thereof; and
(b) measuring the level of bound ANP-SP polypeptide.

In one embodiment, the binding agent is a selective (specific) binding agent. That is, it has low cross-reactivity with other markers of biological events, and more particularly ANP or NT-ANP. The binding agent in one embodiment is an antibody or antigen-binding fragment thereof.

The present invention also relates to such antibodies, or antigen-binding fragments of the antibodies. The antibodies may be in isolated or purified form. An antibody that binds to ANP-SP or a fragment or variant thereof may be in any form, including all classes of polyclonal, monoclonal, single chain, human, humanized antibodies and chimeric antibodies produced by genetic recombination. Also included is antiserum obtained by immunizing an animal such as a mouse, rat or rabbit with ANP-SP or a fragment or variant thereof. The antibodies may bind to a common ANP-SP sequence in a group of ANP-SP fragments, or to a specific ANP-SP fragment, or even to sets of ANP-SP fragments.

A fragment of an antibody or a modified antibody may also be used herein so long as it binds ANP-SP or a fragment or variant thereof. The antigen-binding fragment may be Fab, F(ab′)₂, F(ab′), an Fc or Fv fragment or single chain Fv (scFv), in which Fv fragments from H and L chains are ligated by an appropriate linker (Huston et al. Proc. Natl. Acad. Sci. USA 85:5879-83 (1988)). The “Fe” portion of an antibody refers to that portion of an immunoglobulin heavy chain that comprises one or more heavy chain constant region domains; CH1, CH2 and CH3, but does not include the heavy chain variable region.

The “Fv” portion of an antibody is the minimum antibody fragment that contains a complete antigen-recognition and antigen binding site. The region consists of a dimer of one heavy chain and one light chain variable domain in tight, non-covalent association.

Fab fragments contain the constant domain of the light chain and the first constant domain (CHI) of the heavy chains. Fab′ fragments have a few residues added to the Fab carboxy terminus of the CHI domain including one or more cysteines from the antibody hinge region. F(ab′)₂ fragments represent pairs of Fab′ fragments with cysteine hinges between them, that have been separated. The F(ab′)₂ fragment has two-antigen binding sites. Fab fragments may be produced by papain digestion of antibodies.

For a discussion of antibodies and fragments see for example PNAS USA 81: 6851-6855 (1984), Protein Eng 8(10) 1057-1062 (1995); The Pharmacology of Monoclonal Antibodies, vol. 113, Springer-verlag 1994, Rosenburg and Moore Eds; PNAS USA 90: 6444-6448 (1993); Nature 321: 522-525 (1986); Nature 332: 323-329 (1988), and WO 2005/003154.

Methods for preparing antibodies, and detecting, modifying and isolating same are well known in the art (see for example Maintaining and using Antibodies: A Practical Handbook, Howard, G et al., CRC Press 2006; Protein-protein Interactions: A Molecular Cloning Manual, Golemis E (Ed), CSHL Press, 2002; Harlow and Lane (1998),¹⁰ Milstein¹⁸, Suresh¹⁹, and Brennan²⁰). In one embodiment antibodies used are produced by immunizing a suitable host mammal. Fusion proteins comprising ANP-SP may also be used as immunogens.

An antibody may be modified by conjugation with a variety of molecules, such as polyethylene glycol (PEG), biotin, streptavidin, and chemiluminescent, fluorescent, calorimetrix, and radioimmunometric labels as discussed herein. The modified antibody can be obtained by chemically modifying an antibody. These modification methods are conventional in the field.

Alternatively, an antibody may be obtained as a chimeric antibody, between a variable region derived from nonhuman antibody and the constant region derived from human antibody, or as a humanized antibody, comprising the complementarity determining region (CDR) derived from nonhuman antibody, the frame work region (FR) derived from human antibody, and the constant region. Such antibodies can be prepared using known art methods.^{16,17,22}

In brief, methods of preparing polyclonal antibodies are known to the skilled artisan. Polyclonal antibodies can be raised in a mammal, for example, by one or more injections of an immunizing agent and, if desired, an adjuvant. Typically, the immunizing agent and/or adjuvant will be injected in the mammal by multiple subcutaneous or intraperitoneal injections. The immunizing agent may include ANP-SP or a fragment or variant thereof or a fusion protein thereof. It may be useful to conjugate the immunizing agent to a protein known to be immunogenic in the mammal being immunized. Examples of such immunogenic proteins include but are not limited to keyhole limpet hemocyanin, bovine serum albumin, bovine thyroglobulin, and soybean trypsin inhibitor. Examples of adjuvants which may be employed include Freund's complete adjuvant and MPL TDM adjuvant (monophosphoryl Lipid A, synthetic trehalose dicorynomycolate). The immunization protocol may be selected by one skilled in the art without undue experimentation.

Monoclonal antibodies may be prepared using hybridoma methods well known in the art. See for example Kohler and Milstein, 1975¹¹, U.S. Pat. No. 4,196,265, U.S. Pat. No. 4,816,567 and Golemis (supra). The hybridoma cells may be cultured in a suitable culture medium, alternatively, the hybridoma cells may be grown in vivo as ascites in a mammal. Preferred immortalized cell lines are murine myeloma lines, which can be obtained, for example, from the American Type Culture Collection, Virginia, USA. Immunoassays may be used to screen for immortalized cell lines which secrete the antibody of interest. Sequences of ANP-SP or fragments or variants thereof may be used in screening.

Accordingly, also contemplated herein are hybridomas which are immortalized cell lines capable of secreting a ANP-SP specific monoclonal antibody.

Well known means for establishing binding specificity of monoclonal antibodies produced by the hybridoma cells include immunoprecipitation, radiolinked immunoassay (MA), enzyme-linked immunoabsorbent assay (ELISA) and Western blot. (Lutz et al., Exp. Cell. Res. 175:109-124 (1988), Golemis (supra), and Howard (supra)). For example, the binding affinity of the monoclonal antibody can, for example, be determined by the Scatchard analysis described in Munson et al., Anal Biochem 107: 220 (1980). Samples from immunised animals may similarly be screened for the presence of polyclonal antibodies.

Monoclonal antibodies can also be obtained from recombinant host cells. DNA encoding the antibody can be obtained from a hybridoma cell line. The DNA is then placed into an expression vector, transfected into host cells (eg, COS cells, CHO cells, E. coli cells) and the antibody produced in the host cells. The antibody may then be isolated and/or purified using standard techniques.

Other known art techniques for monoclonal antibody production such as from phage libraries, may also be used. See for example, Nature 352: 624-628 (1991).

To facilitate detection, antibodies and fragments herein may be labelled with detectable markers such as fluorescent, bioluminescent, and chemiluminescent compounds, as well as radioisotopes, magnetic beads and affinity labels (e.g biotin and avidin). Examples of labels which permit indirect measurement of binding include enzymes where the substrate may provide for a coloured fluorescent product, suitable enzymes include horseradish peroxidase, alkaline phosphatase, malate dehydrogenase and the like. Fluorochromes (e.g Texas Red, fluorescein, phycobiliproteins, and phycoerythrin) can be used with a fluorescence activated cell sorter. Labelling techniques are well known in the art.

The monoclonal antibodies secreted by the cells may be isolated or purified from the culture medium or ascites fluid by conventional immunoglobulin purification procedures such as, for example, reverse phase HPLC, protein A-Sepharose, hydroxyapatite chromatography, gel electrophoresis, dialysis, or affinity chromatography. See for example, Scopes, Protein Purification: Principles and Practice, Springer-Verlag, NY (1982).

The monoclonal antibodies or fragments may also be produced by recombinant DNA means (see for example U.S. Pat. No. 4,816,567). DNA modifications such as substituting the coding sequence for human heavy and light chain constant domains in place of the homologous murine sequences (U.S. Pat. No. 4,816,567 above) are also possible. The antibodies may be monovalent antibodies. Methods for preparing monovalent antibodies are well known in the art (U.S. Pat. Nos. 5,334,708, 5,821,047, and 7,476,724). Production of chimeric (U.S. Pat. No. 4,816,567), bivalent antibodies (U.S. Pat. No. 5,843,708) and multivalent antibodies are also contemplated herein (U.S. Pat. No. 6,020,153).

Chimeric monoclonal antibodies are antibodies in which a portion of the heavy and/or light chain is identical with or homologous to corresponding sequences in antibodies derived from a particular species or belonging to a particular antibody (sub)class. The remainder of the chain is identical, or homologous to corresponding sequences in antibodies derived from another species or belonging to another antibody (sub)class, and fragments thereof, so long as they exhibit the requisite biological activity. (See U.S. Pat. No. 4,816,567 supra).

The antibodies of the invention may further comprise humanized antibodies or human antibodies. Humanized antibodies include human immunoglobulins in which residues from a complementary determining region (CDR) of the recipient are replaced by residues from a CDR of a non-human species. The production of humanized antibodies from non-human sources such as rabbit, rat and mouse are well known.^{12,13,14}

Human antibodies can also be produced using various techniques known in the art, including phage display libraries¹⁵; and transgenic methods, see, for example Neuberger 1996¹⁶; and Vaughan et al, 1998¹⁷.

Bispecific antibodies may also be useful. These antibodies are monoclonal, preferably human or humanized, antibodies that have binding specificities for at least two different antigens. For example ANP-SP or a variant or fragment thereof, and an antigen selected from the group including preproANP, ANP, CK-MB, TnT, TnI, BNP, BNP-SP, and myoglobin. Antibodies with greater than two specificities, for example trispecific antibodies, are also contemplated herein.

Methods for making bispecific antibodies are known in the art. See for example Milstein and Cuello 1983¹⁸, Suresh et al., 1986¹⁹ and Brennan et al., 1985²⁰.

The ANP-SP which is selectively bound by the antibody is ANP-SP or a variant or fragment thereof as discussed above.

In one embodiment, the antibody binds the C-terminus (16-25) of ANP-SP. An example of specific antigenic peptide which the binding agent selectively binds is ANP-SP (16-25) SEQ ID NO:12. Another example is ANP-SP (1-10) SEQ ID NO:16.

Binding of ANP-SP can be detected by any means known in the art including specific (antibody based) and non specific (such as HPLC solid phase). Most commonly, antibodies herein are detected using an assay such as ELISA or RIA as noted above. Competitive binding assays, sandwich assays, non-competitive assays, fluoroimmunoassay, immunofluorometric assay, or immunoradiometric assays, luminescence assays, chemiluniescence assays and mass spectrometry analysis such a surface-enhanced laser desorption and ionization (SELDI), electrospray ionization (ESI), matrix assisted laser-desorption ionization WALDO, Fourier transform Ion cyclotron resonance mass spectroscopy (FTICR) alone or in combination with non-specific binding agents such as chromatography formats are also feasible. See for example, Golemis, E and Howard G. (supra).

Conveniently, an antibody can be fixed to a solid substrate to facilitate washing and isolation of the ANP-SP/antibody complex. Binding of antibodies to a solid support can be achieved using known art techniques. See for example Handbook of Experimental Immunology, 4th edition, Blackwell Scientific Publications, Oxford (1986). Useful solid substrates for antibodies include glass, nylon, paper and plastics. Similarly, ANP-SP can be adsorbed onto a solid substrate such as adsorbent silica, or resin particles, or silicon chips optionally coated or derivatised with ion exchange, reverse phase (eg C18 coating) or other materials. The substrate may be in the form of beads, plates, tubes, sticks or biochips. Examples of biochips include Ciphergen, ProteinChip arrays (Ciphergen Biosystems (CA, USA)), and Packard BioChips available from Perkin Elmer, USA. See also U.S. Pat. No. 6,225,047, U.S. Pat. No. 6,329,209. The biochips may include a chromatographic surface. Biochips or plates with addressable locations and discreet microtitre plates are particularly useful. Also preferred for use are multiplex systems where beads containing antibodies directed to multiple analytes are used to measure levels of the analytes in a single sample. Analytes to be measured may include other cardiac markers as well as ANP-SP or variants or fragments thereof. One example of a suitable multiplex bead system for use herein is the Luminex Fluorokine Multianalyte Profiling system (R&D Systems, MN, USA).

Antibody assay methods are well known in the art see for example U.S. Pat. No. 5,221,685, U.S. Pat. No. 5,310,687, U.S. Pat. No. 5,480,792, U.S. Pat. No. 5,525,524, U.S. Pat. No. 5,679,526, U.S. Pat. No. 5,824,799, U.S. Pat. No. 5,851,776, U.S. Pat. No. 5,885,527, U.S. Pat. No. 5,922,615, U.S. Pat. No. 5,939,272, U.S. Pat. No. 5,647,124, U.S. Pat. No. 5,985,579, U.S. Pat. No. 6,019,944, U.S. Pat. No. 6,113,855, U.S. Pat. No. 6,143,576 and for unlabelled assays U.S. Pat. No. 5,955,377, and U.S. Pat. No. 5,631,171 see also Zola, Monoclonal Antibodies: A Manual of Techniques pp 147-158 (CRC Press, Inc 1987), Harlow and Lane (1998) Antibodies, A Laboratory Manual, Cold Spring Harbour Publications, New York, and US 2005/0064511 for a description of assay formats and conditions. All of the above references are incorporated herein by reference in their entirety.

Immunoassay analysers are also well known and include Beckman Access, Abbott AxSym, Roche EleeSys and Dade Behring Status systems amongst others which are well described²¹.

Binding of ANP-SP and an antibody to form a complex can be detected directly or indirectly. Direct detection is carried out using labels such as fluorescence, luminescence, radionuclides, metals, dyes and the like. Indirect detection includes binding detectable labels such as digoxin or enzymes such as horseradish peroxidase and alkaline phosphatase to form a labelled ANP-SP antibody followed by a step of detecting the label by addition of detection reagents.

Horseradish peroxidase for example can be incubated with substrates such as o-Phenylenediamine Dihyhydrochloride (OPD) and peroxide to generate a coloured product whose absorbance can be measured, or with luminol and peroxide to give chemiluminescent light which can be measured in a luminometer as is known in the art. Biotin or digoxin can be reacted with binding agents that bind strongly to them. For example, the proteins avidin and streptavidin will bind strongly to biotin. A further measurable label is then covalently bound or linked thereto either by direct reaction with the protein, or through the use of commonly available crosslinking agents such as MCS and carbodiimide, or by addition of chelating agents.

Generally, the complex is separated from the uncomplexed reagents for example by centrifugation. If the antibody is labelled, the amount of complex will be reflected by the amount of label detected. Alternatively, an ANP-SP may be labelled by binding to an antibody and detected in a competitive assay by measuring a reduction in bound labelled ANP-SP when the antibody-labelled-ANP-SP is incubated with a biological sample containing unlabelled ANP-SP. Other immunoassays may be used for example a sandwich assay.

In one embodiment, following contact with the antibody, usually overnight for 18 to 25 hours at 4° C., or for 1 to 2 to 4 hours at 25° C. to 40° C., the labelled ANP-SP bound to the binding agent (antibody) is separated from the unbound labelled ANP-SP. In solution phase assays, the separation may be accomplished by addition of an anti gamma globulin antibody (second-antibody) coupled to solid phase particles such as cellulose, or magnetic material. The second-antibody is raised in a different species to that used for the primary antibody and binds the primary antibody. All primary antibodies are therefore bound to the solid phase via the second antibody. This complex is removed from solution by centrifugation or magnetic attraction and the bound labelled peptide measured using the label bound to it. Other options for separating bound from free label include formation of immune complexes, which precipitate from solution, precipitation of the antibodies by polyethyleneglycol or binding free labelled peptide to charcoal and removal from solution by centrifugation of filtration. The label in the separated bound or free phase is measured by an appropriate method such as those presented above.

Competitive binding assays can also be configured as solid phase assays that are easier to perform and are therefore preferable to those above. This type of assay uses plates with wells (commonly known as ELISA or immunoassay plates), solid beads or the surfaces of tubes. The primary antibody is either adsorbed or covalently bound to the surface of the plate, bead or tube, or is bound indirectly through a second anti gamma globulin or anti Fc region antibody adsorbed or covalently bound to the plate. Sample and labelled peptide (as above) are added to the plate either together or sequentially and incubated under conditions allowing competition for antibody binding between ANP-SP in the sample and the labelled peptide. Unbound labelled peptide can subsequently be aspirated off and the plate rinsed leaving the antibody bound labelled peptide attached to the plate. The labelled peptide can then be measured using techniques described above.

Sandwich type assays have greater specificity, speed and greater measuring range. In this type of assay an excess of the primary antibody to ANP-SP is attached to the well of an ELISA plate, bead or tube via adsorption, covalent coupling, or an anti Fc or gamma globulin antibody, as described above for solid phase competition binding assays. Sample fluid or extract is contacted with the antibody attached to the solid phase. Because the antibody is in excess this binding reaction is usually rapid. A second antibody to ANP-SP is also incubated with the sample either simultaneously or sequentially with the primary antibody. This second antibody is chosen to bind to a site on ANP-SP that is different from the binding site of the primary antibody. These two antibody reactions result in a sandwich with the ANP-SP from the sample sandwiched between the two antibodies. The second antibody is usually labelled with a readily measurable compound as detailed above for competitive binding assays. Alternatively a labelled third antibody which binds specifically to the second antibody may be contacted with the sample. After washing away the unbound material the bound labelled antibody can be measured and quantified by methods outlined for competitive binding assays.

A dipstick type assay may also be used. These assays are well known in the art. They may for example, employ small particles such as gold or coloured latex particles with specific antibodies attached. The liquid sample to be measured may be added to one end of a membrane or paper strip preloaded with the particles and allowed to migrate along the strip. Binding of the antigen in the sample to the particles modifies the ability of the particles to bind to trapping sites, which contain binding agents for the particles such as antigens or antibodies, further along the strip. Accumulation of the coloured particles at these sites results in colour development are dependent on the concentration of competing antigen in the sample. Other dipstick methods may employ antibodies covalently bound to paper or membrane strips to trap antigen in the sample. Subsequent reactions employing second antibodies coupled to enzymes such as horse radish peroxidase and incubation with substrates to produce colour, fluorescent or chemiluminescent light output will enable quantitation of antigen in the sample.

As discussed in the following examples, in one embodiment radioimmunoassay (RIA) is the laboratory technique used. In one RIA a radiolabelled antigen and unlabelled antigen are employed in competitive binding with an antibody. Common radiolabels include ¹²⁵I, ¹³¹I, ³H and ¹⁴C.

Radioimmunoassays involving precipitation of ANP-SP with a specific antibody and radiolabelled antibody binding protein can measure the amount of labelled antibody in the precipitate as proportional to the amount of ANP-SP in the sample. Alternatively, a labelled ANP-SP is produced and an unlabelled antibody binding protein is used. A biological sample to be tested is then added. The decrease in counts from the labelled ANP-SP is proportional to the amount of ANP-SP in the sample.

In RIA it is also feasible to separate bound ANP-SP from free ANP-SP. This may involve precipitating the ANP-SP/antibody complex with a second antibody. For example, if the ANP-SP antibody complex contains rabbit antibody then donkey anti-rabbit antibody can be used to precipitate the complex and the amount of label counted. For example in an LKB, Gammamaster counter. See Hunt et al.²¹

The methods of the invention further comprise measuring the levels of one or more non-ANP-SP markers of the ACD, cardiac transplant rejection, or ACD/pulmonary disorder. The level of the other marker or markers can be compared to mean control levels from a control population. A deviation in the measured level from the mean control level is predictive or diagnostic of ACD or cardiac transplant rejection.

While the methods of the invention have been described with respect to a higher level or increase in ANP-SP levels being indicative of ACD, or cardiac transplant rejection, it is also possible that in some events or disorders the levels of ANP-SP will fall or be lower. Measuring deviations below a control level are also contemplated.

Other markers which are particularly useful herein include troponin T, troponin I, creatin kinase MB, myoglobin, BNP, NT-BNP, BNP-SP, LDH, aspartate aminotransferase, H-FABP, endothelin, adrenomedullin, rennin and angiotensin II^{I}. These markers are all implicated in cardiac dysfunction or disease. Correlating the level of ANP-SP with other markers can increase the predictive, diagnostic or monitoring value of ANP-SP. In the case of ACD, cardiac transplant rejection or ACD/pulmonary disorder combining ANP-SP marker levels with known cardiac markers can increase the predictive or diagnostic value of a patient outcome.

Analysis of a number of peptide markers can be carried out simultaneously or separately using a single test sample. Simultaneous, two or multi-site format assays are preferred. Multiplex bead, microassay or biochip systems are particularly useful. The beads, assays or chips can have a number of discreet, often addressable locations, comprising an antibody to one or more markers including ANP-SP. The one or more markers include more than one ANP-SP marker. For example, it may be useful to assay for N-terminal and C-terminal ANP-SP fragments and combine the assay results. Many other such marker combinations are feasible. US2005/0064511, U.S. Pat. No. 6,019,944, and Ng and Ilang, J. Cell Mal. Med., 6:329-340 (2002) provide a description of microarray, chips, capillary devices and techniques useful in the present invention. Luminex provides a multiplex bead system useful in the present invention. See also The Protein Protocols Handbook, supra. Laboratory analysers suitable for use with separate or sequential assays include AxSym (Abbott, USA), ElecSys (Roche), Access (Beckman), ADVIA CENTAUR® (Bayer) and Nichols Advantage® (Nichols Institute) immunoassay system.

In one embodiment simultaneous assays of a plurality of polypeptides are performed on a single surface such as a chip or array.

Where a subject is to be monitored, a number of biological samples may be taken over time. Serial sampling allows changes in marker levels, particular ANP-SP to be measured over time. Sampling can provide information on the approximate onset time of an event, the severity of the event, indicate which therapeutic regimes may be appropriate, response to therapeutic regimes employed, or long term prognosis. Analysis may be carried out at points of care such as in ambulances, doctors offices, on clinical presentation, during hospital stays, in outpatients, or during routine health screening.

The methods of the invention may also be performed in conjunction with an analysis of one or more risk factors such as but not limited to age, weight, sex and family history of events such as cardiac events. Test results can also be used in conjunction with the methods of the invention. For example, ECG results and clinical examination. A statistically significant increase in circulating level of ANP-SP, together with one or more additional risk factors or test results may be used to more accurately diagnose or prognose the subject's condition.

The methods herein can also be used as a guide to therapy. For example what therapies to initiate and when, therapy monitoring, detection of positive or adverse effects of therapy, for example heart toxicity of antimitotic drugs, and adjustment of therapeutic regimes if and when required dependent on results. This can improve short, medium and long term outcomes for patients. For a guide to treatments see Troughton et al.²⁴

### Acute Cardiac Disorders

The applicants have shown that concentrations of the full-length ANP-SP molecule (1-25) and various fragments thereof are correlated with acute cardiac disorders. Moreover, ANP-SP levels are at their highest upon clinical presentation in the case of patients presenting with suspected acute myocardial infarction (AMI) or heart attack. Patients presenting with acute cardiac disorders, and in particular acute cardiac ischemia coronary artery disease caused by (heart attack leaving scarring in the heart muscle or myocardium) may or may not experience subsequent myocardial infarction (MI). The group which does not experience MI can not be readily diagnosed using current clinical techniques and markers. For the first time, the applicants have therefore provided a useful early and specific marker for myocardial damage associated with MI. This may allow the early diagnosis of myocardial damage due to adverse events (AEs) and allow a physician to distinguish such cases from other acute coronary syndromes as well as from other causes of a chest pain. For example angina, gastro-intestinal disease, lung/pleural disorders and the like. This significantly shortens the window of 6 hours to 12 hours currently experienced waiting for elevation of levels of current cardiac biomarkers such as myoglobin, CK-MB, TnT and TnI. A more precise diagnosis and treatment can therefore be effected earlier, reducing morbidity and mortality and giving better prognostic outcomes.

In another embodiment, the invention has application in monitoring reperfusion treatment in cardiac patients. Reperfusion treatment commonly includes percutaneous coronary intervention (eg angioplasty) and/or pharmacological treatment. Thrombolytic drugs for revascularisation are commonly employed in pharmacological treatment. Adjunctive therapies include anticoagulant and anti-platelet therapies. Reperfusion treatment is most effective when employed as soon as possible after diagnosis. ANP-SP testing to accelerate diagnosis allows prompt introduction of reperfusion treatment. Effectiveness of treatment can also be monitored by repeat testing, and therapy adjusted as appropriate. For a comprehensive discussion of reperfusion treatment see Braunwald et al herein¹.

### Cardiac Disease

The methods of the invention may also be useful to diagnose or predict cardiac disease in a subject.

The applicants have shown that in patients with acute cardiac disorders the levels of ANP-SP are elevated after a cardiac event. It is similarly possible that patients with cardiac disease or at risk of same will exhibit a higher level of ANP-SP than mean control levels in a control population. This suggests ANP-SP has broad applications as a marker of cardiac disease.

### Cardiac Transplant Rejection

The invention also has applications in monitoring heart transplant, commonly a cardiac allograft transplant, rejection through regular tissue biopsy during and after transplant using ANP-SP measurements. An increase in ANP-SP levels measured within four or two hours of heart transplant relative to a control level may be predictive or diagnostic of a rejection episode.

The present invention also provides an assay for ANP-SP in a biological sample obtained from a subject within four or two hours from onset of, or within four or two hours of clinical presentation with ACD, cardiac transplant rejection or ACD/pulmonary disorder, the assay comprising detecting and measuring the level of ANP-SP in the sample using any known methods. Preferably, the assay is an in vitro assay. Such methods include all of the known assay techniques discussed above as well as gel electrophoresis techniques, Western blot, gas phase spectroscopy, atomic force microscopy, surface plasmon resonance, mass spectroscopy but not limited thereto²².

In one embodiment the assay comprises one or more nucleic acid sequences which bind to one or more of the ANP-SP nucleic acid sequences of the invention. A large range of sense and antisense probes and primers can be designed from the nucleic acid sequences herein. The expression level of the ANP-SP sequence is identified using known art techniques discussed above. The array can be a solid substrate e.g., a “chip” as described in U.S. Pat. No. 5,744,305 or a nitrocellulose membrane. For a discussion of useful arrays see for example Microarray Technology and its Application, Müller, U et al., Springer 2005, and Gene Expression Profiling by Microarrays: Clinical Implications, Hofmann, W-K; Cambridge University Press 2006.

Proteins expressed by the ANP-SP marker herein may also be used in assays, and results compared to expression levels of the same protein expressed in a normal control sample. Protein presence and quantity may be assessed using assay formats known in the art and discussed herein.

The presence of ANP-SP is preferably detected in the sample by binding ANP-SP to a binding agent such as an antibody, including an antibody of the invention and measuring the presence of the amount of bound ANP-SP.

As noted above, antibodies selective for ANP-SP including variants and fragments thereof form a further aspect of the invention and the antibodies may be prepared by the techniques discussed above. The antibodies are useful in the methods and assay of the invention.

In a further aspect, the invention provides a kit for predicting, diagnosing or monitoring acute cardiac disorder (ACD), cardiac transplant rejection, or ACD/pulmonary disorder, comprising an ANP-SP binding agent including an antibody or antigen-binding fragment of the invention. In one embodiment, the kit is for use with a biological sample obtained from a subject within four or two hours of onset of, or clinical presentation with ACD, cardiac transplant rejection, or ACD/pulmonary disorder. Known art ANP binding agents may also be useful in the kit.

The invention also provides a kit for predicting, diagnosing or monitoring an acute cardiac disorder (ACD), cardiac transplant rejection, or an ACD/pulmonary disorder comprising a binding agent of the invention, wherein the kit is calibrated to measure ANP-SP levels in the range of 0.1 to 500 μmol/L, or Ito 300 pmol/L, or 2 to 100 pmol/L, or 5 to 150 pmol/L.

Calibration of assays can be effected according to known art techniques, for example using blood samples with known levels of ANP-SP, or a set of calibrates with different known levels of ANP-SP in each. Test strips for use in diagnostic kits are commonly calibrated during manufacture. See for example U.S. Pat. No. 6,780,645. The kit is useful for measuring the level of ANP-SP in a biological sample. The detection reagents may be oligonucleotide sequences complementary to ANP-SP or a fragment of the ANP-SP marker, or antibodies which bind to the polypeptides encoded by the marker. The reagents may be bound to a solid matrix as discussed above or packaged with reagents for binding them to the matrix. The solid matrix or substrate may be in the form of beads, plates, tubes, dip sticks, strips or biochips all as discussed above.

Detection reagents include wash reagents and reagents capable of detecting bound antibodies (such as labelled secondary antibodies), or reagents capable of reacting with the labelled antibody.

The kit will also conveniently include a control reagent (positive and/or negative) and/or a means for detecting the nucleic acid or antibody. Instructions for use may also be included with the kit, such as taking a biological sample from a subject within four or two hours of onset or presentation with ACD, cardiac transplant rejection or ACD/pulmonary disorder, measuring the level of ANP-SP in the sample, comparing same to a control level and associating the result with cardiac status. Generally an increase in the ANP-SP marker level from a control is indicative of ACD or cardiac transplant rejection, or ACD as opposed to a pulmonary disorder.

Most usually, the kits will be formatted for assays known in the art, and in one embodiment for PCR, Northern hybridization or Southern ELISA assays, as are known in the art.

The kits may also include one or more additional markers for ACD, transplant rejection, or ACD/pulmonary disorders. In the case of ACS the additional marker may include one or more of troponin T, troponin 1, creatin kinase MB, myoglobin, BNP, NT-BNP, BNP-SP, LDH, aspartate aminotransferase, H-FABP, endothelin, adrenomedullin, rennin and ongrotensin II. In one embodiment all of the markers are included in the kit.

The kit will be comprised of one or more containers and may also include collection equipment, for example, bottles, bags (such as intravenous fluids bags), vials, syringes, and test tubes. At least one container holds a product which is effective for predicting, diagnosing, or monitoring ACD (particularly ACS), transplant rejection, or ACD/pulmonary disorder. The product is usually a nucleic acid molecule, polypeptide or a binding agent, particularly an antibody or antigen-binding fragment of the invention, or a composition comprising any of these. In a preferred embodiment, an instruction or label on, or associated with, the container indicates that the composition is used for predicting, diagnosing, or monitoring ACD (particularly ACS), transplant rejection, or ACD/pulmonary disorders. Other components may include needles, diluents and buffers. Usefully, the kit may include at least one container comprising a pharmaceutically-acceptable buffer, such as phosphate-buffered saline, Ringer's solution and dextrose solution.

Binding agents that selectively bind ANP-SP are desirably included in the kit. In one embodiment, the binding agent is an antibody, preferably an antibody or antigen-binding fragment of the invention. The antibody used in the assays and kits may be monoclonal or polyclonal and may be prepared in any mammal as discussed above. The antibodies may be prepared against a native peptide encoded or indicated by a ANP-SP nucleic acid sequence of the invention, ANP-SP (1-25), or a synthetic peptide based on same, or may be raised against an exogenous sequence fused to a nucleic acid sequence encoding a ANP-SP peptide of the invention.

In one kit embodiment a ANP-SP detection reagent is immobilized on a solid matrix such as a porous strip or chip to form at least one ANP-SP detection site. The measurement or detection region of the porous strip may include a plurality of detection sites, such detection sites containing a ANP-SP detection reagent. The sites may be arranged in a bar, cross or dot or other arrangement. A test strip or chip may also contain sites for negative and/or positive controls. The control sites may alternatively be on a different strip or chip. The different detection sites may contain different amounts of immobilized nucleic acids or antibodies eg, a higher amount in the first detection site and lower amounts in subsequent sites. Upon the addition of a test biological sample the number of sites displaying a detectable signal provides a quantitative indication of the amount of ANP-SP present in the sample.

Also included in the kit may be a device for sample analysis comprising a disposable testing cartridge with appropriate components (markers, antibodies and reagents) to carry out sample testing. The device will conveniently include a testing zone and test result window. Immunochromatographic cartridges are examples of such devices. See for example U.S. Pat. No. 6,399,398; U.S. Pat. No. 6,235,241 and U.S. Pat. No. 5,504,013.

Alternatively, the device may be an electronic device which allows input, storage and evaluation of levels of the measured marker against control levels and other marker levels. US 2006/0234315 provides examples of such devices. Also useful in the invention are Ciphergen's Protein Chip® which can be used to process SELDI results using Ciphergen's Protein Chip® software package.

In this specification where reference has been made to patent specifications, other external documents, or other sources of information, this is generally for the purpose of providing a context for discussing the features of the invention. Unless specifically stated otherwise, reference to such external documents is not to be construed as an admission that such documents; or such sources of information, in any jurisdiction, are prior art, or form part of the common general knowledge in the art.

The invention will now be illustrated in a non-limiting way be reference to the following examples.

### Example 1

### Methods

All human protocols were approved by the Upper South Regional Ethics Committee of the Ministry of Health, New Zealand and were performed in accord with the Declaration of Helsinki.

### Chemicals

Synthetic human ANP signal peptide ANP-SP (16-25) (SEQ ID NO:12) and ANP-SP (1-10) (SEQ ID NO:16) was synthesised by Mimotopes (Australia) using a mild Fmoc Solid Phase Synthesis method²⁹. All buffer reagents were purchased from BDH® (UK) and/or Sigma (Mo, USA). ANP-SP (16-25) was synthesised with the C-terminal extended with cysteine for directional carrier coupling. ANP-SP (16-25) was also C-terminally extended with a tyrosyl residue for tracer preparation on the same peptide.

### Human Studies

For the healthy volunteer reference range study, blood samples were initially obtained from 8 healthy volunteers (5 woman, 3 men average age 47±-8 years (range 31-65 years), BMI 25.1±3.2 kg/m²) after an overnight fast.

The study was subsequently extended to 66 healthy volunteers, average age 47±8 years (range 31-65 years), BMI 25.1±3.2 kg./m². Blood samples were also obtained from this group after overnight fast.

For analysis of ANP-SP concentrations in acute cardiac injury, we initially studied 3 consecutive patients (3 men, average age 67±3 years (range 65-70 years)), presenting to the Coronary Care Unit at Christchurch Hospital within 4 hours of the onset of chest pain and clear evidence of ST-elevation acute MI, together with a rise then fall in plasma troponin T (TnT). Patients with cardiogenic shock were excluded. The study was subsequently extended to include 23 patients, average age 67±3 years (range 65-70 years). BMI not measured.

An 18-gauge intravenous cannula was inserted into a forearm vein for blood sampling. Venous samples (10 ml) were drawn on admission to the Coronary Care Unit (time 0) and thereafter at 0.5, 1, 4, 8, 12, 24 and 72 h as in-patients. Samples were taken into tubes on ice and centrifuged at +4° C. at 2700 g for 5 min and the plasma stored at −80° C. until analysed.

### Plasma Extraction

All plasma samples were extracted on SepPak Cartridges, (Waters, USA) as previously described²¹, dried and stored at −20° C. prior to RIA and HPLC.

### Hormone Concentration Analysis

Plasma samples were assayed for TnT, CK-MB and myoglobin using heterogeneous immunoassays on an Elecsys 2010 (Roche, USA) using ruthenium-labelled biotinylated antibodies according to standard manufacturers' protocols, Roche Diagnostics.¹⁷ ANP-SP was measured by specific RIA as follows:

### ANP-SP MA

For the measurement of putative human ANP-SP IR peptides, we generated a novel and specific RIA directed against amino acids 16-25 of the human preproANP(1-25) signal sequence (SEQ ID NO:1).

### Antibody Generation

preproANPCys¹⁵ (16-25) was coupled to malemide treated N-e-maleimidocaproyloxy succinimide ester (EMCS) derivatised BSA in PBS (pH 7.0) by gentle mixing at room temperature. Coupled peptide was emulsified with Freund's adjuvant (2 ml) and injected subcutaneously (2 ml total) in 2 New Zealand white rabbits over 4-5 sites at monthly intervals. Rabbits were bled 12 days after injection to assess antibody titres until adequate levels were achieved. For RIA, ANP-SP IR was determined using antiserum at a final dilution of 1:15,000. This antiserum has no detectable cross reactivity with peptides and drugs indicated in FIG. 5 including human proBNP(1-13), proBNP(1-76), proANP(1-30), ANP, ANP-SPn (1-10), BNP, BNP-SPn (1-10), BNP-SPc (17-26), endothelin 1, Angiotensin II, Angiotensin(1-7), urotensin II, CNP, proCNP(1-15), adrenomedullin, urocortin I and urocortin II (all ≦0.01%). Cross reactivity was assessed following Klee G G. Interference in hormone immunoassays Clin Lab Med, 2004, 24:1-18.

### Iodination and Assay Method

preproANP Tyr'⁵ (16-25) was iodinated via the Chloramine T method and purified on reverse phase HPLC(RP-HPLC) as previously described²¹. From this preparation two iodinated tracer forms after RP-HPLC were tested. All samples, standards, radioactive traces and antiserum solutions were diluted in potassium based assay buffer.²¹ The assay incubate consisted of 100 μL sample or standard (0-640 pmol human preproANP(16-25) combined with 100 μL antiserum which was vortexed and incubated at 4° C. for 24 hours. 1004, of trace 1 or trace 2 (4000-5000 cpm) was then added and further incubated for 24 hours at 4° C. Free and bound immunoreactivities were finally separated by solid phase second antibody method (donkey anti-rabbit Sac-Cel®, IDS Ltd, England) and counted in a Gammamaster counter (LKB, Uppsala, Sweden).

### Statistical Analysis

All results are presented as mean±SEM. Time-course data were analysed using two-way ANOVA for repeated measurements followed by least significant difference post-hoc testing. Correlation analysis of plasma hormone concentrations was carried out using a general linear regression model. In all analyses, a P-value<0.05 was considered significant.

### Results

To determine if the 25 amino acid signal peptide of ANP, or fragments derived from it, are present in circulation of humans, we developed a specific radioimmunoassay (RIA) directed against residues 16-25 of preproANP(1-25) (ANP-SP, FIG. 2). Dilution of plasma extracts demonstrate parallelism with the standard curve (FIG. 3) and plasma concentrations of ANP-SP in healthy humans were 2.3±0.7 pmol/L (n=8) when trace 1 was used.

Having established that immunoreactive (IR) ANP-SP peptides are present in human plasma we then measured serial concentrations of IR ANP-SP in patients with documented AMI again using trace 1 (n=3, FIG. 4A). Highest concentrations of IR ANP-SP were observed 1-2 hours after hospital admission and slowly dropped to stable levels over 72 hours. Importantly, average peak levels were 5 to 15-fold higher (range 3-7) than levels in normal healthy volunteers and remained higher up to 72 hours. Peak concentrations of myoglobin occurred 1-2 hours after hospital admission, whereas peak TnT and CK-MB levels were not attained until 8-12 hours after admission.

We re-examined the above findings and ratios using the ANP-SP(16-25) RIA based on trace 2. Accordingly, we found that plasma concentrations of ANP-SP in healthy humans using trace 2 were 20.8±5.7 pmol/L (n=66). Following on from this we re-measured serial ANP-SP plasma concentrations in 23 patients with documented AMI using the trace 2 assay. Again, highest concentrations of ANP-SP were observed 1-2 hours after hospital presentation (P<0.001, n=23) which slowly dropped to baseline over 72 hours. Average peak levels were 5-15 fold higher than normal volunteer levels (range 3-8) and these peaks occurred well before those of CK-MB and troponin.

### Example 2

Eight patients with clinically stable suspected ACS were catheterized and blood samples from multiple organ sites: these were the femoral artery (FA), hepatic vein (HV, inferior vena cava (IVC), cardiac coronary sinus vein (CS) and pulmonary artery (PA). Blood was collected into chilled EDTA tubes, prepared from plasma by centrifugation and the plasma submitted to ANP-SP RIA. FIG. 6 clearly shows that the highest site of ANP-SP concentration is the CS, the vein draining the heart, especially the ventricles. This is strong evidence that the heart is the predominant site of ANP-SP secretion and is consistent with the known gene expression pattern of ANP, being highest in the heart.

### CONCLUSION

Circulating ANP-SP concentrations in clinically stable patients are derived from cardiac sources. The significant cardiac secretion, is consistent with ANP-SP being a cardiac hormone.

### Discussion

This evidence is the first to document the signal peptide of preproANP as being present in the circulation and extracellular space within two hours of a patient presenting with ACD or within two hours of the onset of ACD. We show in the first instance that the measurement of ANP-SP in blood has potential as a rapid biomarker of acute cardiac ischemia and/or subsequent injury and in the second instance, that measurement of ANP-SP after the event has potential merit as a marker of long term prognosis and outcome.

Those skilled in the art will of course appreciate that the above description is provided by way of example and that the invention is not limited thereto.

### REFERENCES

1. Braunwald E, Zipes D P, Libby P. Acute myocardial infarction Chp. 35 Heart disease: a textbook of cardiovascular medicine, 6^{th} ed. 2001. pgs. 1114-1231.
2. Tapanainen J M, Lindgren K S, Mäkikallio T H, Vuolteenaho O, Leppäluoto J, Huikuri H V. Natriuretic peptides as predictors of non-sudden and sudden cardiac death after acute myocardial infarction in the beta-blocking era. J Am Coll Cardiol. 2004 43(5):757-763.
3. Jernberg T, Stridsberg M, Venge P, Lindahl B. N-terminal pro Brain Natriuretic Peptide on admission for early risk stratification of patients with chest pain and no ST-segment elevation. J. Am. Coll. Cardiology 2002 40:437-445.
4. Omland T, Persson A, Ng L, O'Brien R, Karlsson T, Herlitz J, Hartford M, Caidahl K. N-terminal pro-B-type natriuretic peptide and long-term mortality in acute coronary syndromes. Circulation. 2002 106:2913-2918.
5. Thibault G, Murthy K K, Gutkowska J, Seidah N G, Lazure C, Chrétien M, Cantin M. NH2-terminal fragment of rat pro-atrial natriuretic factor in the circulation: identification, radioimmunoassay and half-life. Peptides. 1988 9:47-53.
6. Omland T, Aakvaag A, Bonarjee V V, Caidahl K, Lie R T, Nilsen D W, Sundstjord J A, Dickstein K. Plasma brain natriuretic peptide as an indicator of left ventricular systolic function and long-term survival after acute myocardial infarction. Comparison with plasma atrial natriuretic peptide and N-terminal proatrial natriuretic peptide. Circulation. 1996 93(11):1963-1969.
7. Squire I B, O'Brien R J, Demme B, Davies J E, Ng L L. N-terminal pro-atrial natriuretic peptide (N-ANP) and N-terminal pro-B-type natriuretic peptide (N-BNP) in the prediction of death and heart failure in unselected patients following acute myocardial infarction. Clin Sci (Lond). 2004 107(3):309-316.
8. Chenna R, Sugawara H, Koike T, Lopez R., Gibson T J, Higgins D G, Thompson J D. Multiple Sequence Alignment with the Clustal series of programs Nucleic Acids Res (2003) 31 (13): 3497-500.
9. Bowie, J. U et al., (1990). Deciphering the message in Protein Sequences: Tolerance to Amino Acid Substitutions. Science 247, 1306-1310.
10. Harlow and Lane 1998. Antibodies: A Laboratory Manual, Cold Spring Harbour Press New York.²⁷
11. Kohler and Milstein 1975. Continuous cultures of Fused Cells Secreting Antibody of Predefined Specificity. Nature, 256 (5517), 495-497.
12. Verhoeyen M. C Milstein, and G Winter Reshaping human antibodies: grafting an antilysozyme activity. Science 1988 Mar. 25; 239(4847):1534-6.
13. Jones, P. T., Dear, P. H., Foote, J., Neuberger, M. S, and Winter, G. “Replacing the complementarity-determining regions in a human antibody with those from a mouse.” Nature (1986) 321: 522-525.
14. Riechmann L, Clark M, Waldmann H, Winter G. Reshaping human antibodies for therapy. Nature. 1988 Mar. 24; 332(6162):323-7.
15. Hoogenboom H R, Winter G (1992) Human antibodies from synthetic repertoires of germline VH gene segments rearranged in vitro. J Mol. Biol. 1992 Sep. 20; 227 (2):381-8.
16. Michael Neuberger (1996) Generating high-avidity human Mabs in mice Nature Biotechnology 14, 826
17. Tristan J. Vaughan, Jane K. Osbourn & Philip R. Tempest (1998) Human antibodies by design. Nature Biotechnology 16, 535-539
18. Milstein and Cuello (1983) The co-expression of two immunoglobulin heavy-chain/light-chain pairs, where the two heavy chains have different specificities, Nature, 305:537-539.
19. Suresh, M. R., Cuello, A. C. and Milstein, C. (1986) Bi-specific monoclonal antibodies from hybrid hybridomas. Methods in Enzymology, 121: 210-228.
20. Brennan et al., “Preparation of bispecific antibodies by chemical recombination of monoclonal immunoglobulin G1 fragments” Science 229:81-83 (1985).
21. Hunt P J, Richards A M, Nicholls M G, Yandle T G, Doughty R N, Espiner E A. Immunoreactive amino terminal pro brain natriuretic peptide (NT-proBNP): a new marker of cardiac impairment. Clin. Endocrinol. 1997 47:287-296.
22. The Immunoassay Handbook. 3^{rd} edition, ed. David Wild. Elsevier Ltd, 2005.
23. Solber H. Approved recommendation (1987) on the theory of reference values. Part 5. Statistical treatment of collected reference values. Determination of reference limits. Journal of clinical Chemistry and Clinical Biochemistry 1987 25:645-656.
24. Troughton R W, Prior D L, Pereira J J, Martin M, Fogarty A, Morehead A, Yandle T G, Richards A M, Starling R C, Young J B, Thomas J D, Klein A L. Plasma B-type natriuretic peptide levels in systolic heart failure: importance of left ventricular diastolic function and right ventricular systolic function. J Am Coll Cardiol. 2004 43:416-422.
25. Universal definition of myocardial infarction. Consensus statement from the Joint ESC/ACCF/AHA/WHF Taskforce for the redefinition of myocardial infarction. Circulation 2007 116:2634-2653.
26. National Academy of Clinical Biochemistry and IFCC Committee for standardisation of markers of cardiac damage laboratory medicine practice guidelines: analytical issues for biochemical markers of acute coronary syndromes. Circulation 2007 115:e352-e355.
27. Kunkel, Thomas A. Rapid and efficient site-specific mutagenesis without phenotypic selection. Proc. Natl. Acad. Set. USA Vol. 82, pp. 488-492, January 1985.
28. Techniques in Protein Modification By Roger L. Lundblad Edition: 2 Published by CRC Press, 1995 288 pages.
29. Atherton et al. (1989) Solid Phase Synthesis: a practical approach, IRL press.

All references and citations in this list and throughout the specification including patent specifications are hereby incorporated in their entirety.

1. An antibody or antigen fragment binding thereof which binds
(a) ANP-SP amino acid sequence 16-25 (SEQ ID NO:12) or 1-10 (SEQ ID NO:16);
(b) an amino acid sequence encoded by a nucleotide sequence selected from SEQ ID NO:13 or SEQ ID NO:17; or
(c) a variant or fragment of (a) or (b).

2. An antibody or antigen-binding fragment as claimed in claim 1 which selectively binds ANP (16-25) or ANP (1-10).

3. An antibody or antigen-binding fragment as claimed in claim 1, or claim 2 which is a monoclonal, polyclonal, chimeric or humanized antibody or fragment.

4. An antibody or antigen-binding fragment as claimed in any one of claims 1 to 3 which is labelled with a detectable marker.

5. A method for predicting, diagnosing or monitoring an acute cardiac disorder (ACD) in a subject, the method comprising:
measuring the level of ANP-SP in a biological sample obtained from the subject within four hours of onset of the ACD, or within four hours of presentation with ACD; and
comparing the level of said ANP-SP with the ANP-SP level from a control wherein a measured level of ANP-SP higher than the control level is indicative of ACD.

6. A method for monitoring a response to treatment of an acute cardiac disorder (ACD) in a subject, the method comprising measuring the level of ANP-SP in a biological sample obtained from the subject within four hours of onset of the ACD, or within four hours of presentation with the ACD; and comparing the level of said ANP-SP with the ANP-SP level from a control, wherein a change in the measured level of ANP-SP from the control level is indicative of a response to the treatment.

7. A method for predicting, diagnosing or monitoring a cardiac transplant rejection episode in a subject, the method comprising measuring the level of ANP-SP in a biological sample obtained from a subject within four hours of heart transplant and comparing the level of said ANP-SP with the ANP-SP level from a control, wherein a measured level of ANP-SP higher than the control level is indicative of transplant rejection.

8. A method of distinguishing between a pulmonary disorder and an acute cardiac disorder (ACD) in a subject, the method comprising measuring the level of ANP-SP in a biological sample obtained from a subject within four hours of presentation with the disorder; and comparing the level of said ANP-SP with the ANP-SP level from a control wherein a measured ANP-SP level higher than the control level is indicative of ACD.

9. A method for predicting, diagnosing or monitoring an acute cardiac disorder (ACD), cardiac transplant rejection, or ACD/pulmonary disorder in a subject, the method comprising measuring the level of ANP-SP in a biological sample obtained from the subject within the first four hours of onset of, or clinical presentation with ACD, cardiac transplant rejection or ACD/pulmonary disorder, wherein the measured level of ANP-SP is compared with the ANP-SP level from a control wherein a measured level of ANP-SP higher than the control level is indicative of ACD or transplant rejection.

10. A method as claimed in claim any one of claims 5 to 9 wherein the level of ANP-SP is measured within the first two hours, one hour, or 30 minutes of onset of ACD, or clinical presentation with ACD, cardiac transplant rejection, or ACD/pulmonary disorder.

11. A method as claimed in any one of claims 5 to 10 wherein a repeat measurement is made within four to six hours of onset or clinical presentation, or two to three hours of initial measurement.

12. A method as claimed in any one of claims 5 to 11 wherein a level of ANP-SP in the sample in the range 40 to 300 pmol/L, or 42 to 200 pmol/L, or 45 to 200 pmol/L or 45 to 150 pmol/L is indicative of ACD, or cardiac transplant rejection, or distinguishes ACD from a pulmonary disorder.

13. A method as claimed in any one of claims 5 to 11 wherein a level of ANP-SP in the sample which is three to eight times higher than the control level is indicative of ACD, or cardiac transplant rejection, or distinguishes ACD from a pulmonary disorder.

14. A method as claimed in any one of claims 5 to 13 wherein the ACD is selected from the group consisting of the acute coronary syndromes: AMI with ST-elevation on presenting ECG, unstable angina, and non. ST-elevated MI; cardiac ischemia, acute cardiac injury, acute cardiac damage resulting from acute drug toxicity, acute cardiomyopathies, and cardiac transplant rejection.

15. A method as claimed in claim 14 wherein the ACD is non-ST elevated MI.

16. A method as claimed in claim 14 wherein the ACD is acute cardiac ischemia.

17. A method as claimed in any one of claims 5 to 16 wherein the biological sample is a blood, plasma, serum, saliva, interstitial fluid, urine or heart tissue sample.

18. A method as claimed in any one of claims 5 to 17 wherein the measuring step comprises
(a) binding ANP-SP with a binding agent; and
(b) measuring the level of bound ANP-SP.

19. A method as claimed in any one of claims 1 to 18 wherein the binding agent is an antibody or antigen-binding fragment thereof.

20. A method as claimed in claim 19 wherein the ANP-SP to which the antibody binds or selectively binds is ANP-SP (SEQ ID NO:14) or an antigenic fragment or variant thereof.

21. A method as claimed in claim 19 or claim 20, wherein the antibody binds at the N-terminus or C-terminus of ANP-SP.

22. A method as claimed in any one of claims 19 to 21 wherein the antibody or antigen-binding fragment is an antibody or fragment as claimed in any one of claims 1 to 4.

23. A method as claimed in any one of claims 18 to 22 wherein binding of ANP-SP is measured using antibodies or antigen-binding fragments that are immobilised on a solid phase.

24. A method as claimed in any one of claims 5 to 23 wherein the level of ANP-SP is measured using an assay selected from RIA, ELISA, mass spectroscopy, fluoroimmunoassay, immunofluorometric assay, and immunoradiometric assay.

25. A method as claimed in any one of claims 5 to 23 wherein the level of ANP-SP is measured using mass spectroscopy.

26. The method of claim 25 wherein the mass spectroscopy is SELDI, ESI, MALDI or FTICR.

27. A method as claimed in any one of claims 5 to 26 which further comprises measuring the level of one or more non-ANP-SP markers of said ACD, or cardiac transplant rejection, or ACD/pulmonary disorder and comparing the levels against marker levels from a control wherein a deviation in the measured level from the control level, together with a measured level of ANP-SP which is higher than the control level of ANP-SP, is predictive or diagnostic of the ACD, or can be used to monitor said ACD, cardiac transplant rejection or ACD/pulmonary disorder.

28. A method as claimed in claim 27 wherein the non-ANP-SP markers are selected from the group consisting of troponin T, troponin I, creatine kinase-MB, myoglobin, BNP, NT-BNP, BNP-SP, LDH, aspartate aminotransferase and H-FABP.

29. A method as claimed in claim 27 or claim 28 wherein the deviation in measured level from the control level comprises a higher measured level of the non-ANP-SP marker.

30. A method as claimed in any one of claims 5 to 29 wherein the monitoring is monitoring of a response to reperfusion treatment.

31. An assay for ANP-SP in a biological sample obtained from a subject within four hours from onset of ACD, cardiac transplant rejection, or ACD/pulmonary disorder or within four hours of clinical presentation with ACD, cardiac transplant rejection, or ACD/pulmonary disorder, the assay comprising detecting and measuring the level of ANP-SP in the sample using any known methods.

32. An assay as claimed in claim 31 wherein the level of ANP-SP is detected in the sample through binding ANP-SP to a binding agent which binds or selectively binds ANP-SP.

33. An assay for ANP-SP comprising:
(a) binding one or more ANP-SP polypeptides from a biological sample, wherein the ANP-SP polypeptide is selected from the group ANP-SP (1-10) (SEQ ID NO:16), and ANP-SP 16-25 (SEQ ID NO:12), or a variant or fragment thereof; and
(b) measuring the level of bound ANP-SP polypeptide.

34. An assay as claimed in claim 33 wherein the ANP-SP polypeptide is bound using an ANP-SP binding agent, or an antibody or antigen-binding fragment thereof as claimed in any one of claims 1 to 4.

35. An assay as claimed in any one of claims 31 to 34 wherein the level of ANP-SP is measured using mass spectrometry.

36. An assay as claimed in claim 35 wherein the mass spectrometry is SELDI, ESI, MALDI or FTICR.

37. An assay of claim 36 wherein the measuring comprises providing a SELDI probe comprising an ANP-SP antibody or antigen-binding fragment attached to a substrate; contacting the antibody or fragment with the biological sample such that the antibody captures one or more ANP-SP polypeptides from the sample; and measuring the level of bound ANP-SP using SELDI.

38. An assay as claimed in claim 37 wherein the SELDI is performed using a SELDI biochip with a chromatographic surface.

39. An assay as claimed in any one of claims 31 to 34 wherein the level of ANP-SP is measured using an assay selected from RIA, ELISA, immunofluorometric assay and immunoradiometric assay.

40. An ANP-SP binding agent that binds ANP-SP (SEQ ID NO:14) or a fragment or variant thereof for use in predicting, diagnosing or monitoring an acute cardiac disorder (ACD), cardiac transplant rejection or ACD/pulmonary disorder in a subject, wherein the ACD, cardiac transplant rejection or ACD/pulmonary disorder is characterised by the appearance of ANP-SP in a biological sample obtained from the subject within four hours of onset of, or within four hours of clinical presentation with ACD, cardiac transplant rejection or ACD/pulmonary disorder.

41. An ANP-SP binding agent as claimed in claim 40 wherein ANP-SP is present in the sample in the range of from 40 to 300 pmol/L, or 42 to 200 pmol/L, or 45 to 200 pmol/L or 45 to 150 pmol/L.

42. An ANP-SP binding agent as claimed in claim 40 wherein ANP-SP is present in the sample at a level of from three to seven times higher than the mean control of ANP-SP.

43. An ANP-SP binding agent as claimed in any one of claims 40 to 42 which is an antibody or antigen-binding fragment as claimed in any one of claims 1 to 4.

44. A use of ANP-SP binding agent in the manufacture of a prognostic, diagnostic or monitoring tool for assessing an acute cardiac disorder (ACD), cardiac transplant rejection or ACD/pulmonary disorder in a subject, wherein assessment is carried out within four hours of onset of, or within four hours of clinical presentation with ACD, cardiac transplant rejection or ACD/pulmonary disorder.

45. A use of claim 44 wherein the prognostic, diagnostic or monitoring tool is calibrated to measure ANP-SP levels in the range of from 0.1 to 500 pmol/L, or 1 to 300 pmol/L or 2 to 100 pmol/L, or 5 to 150 pmol/L.

46. A use of claim 44 wherein the prognostic, diagnostic or monitoring tool is calibrated to measure ANP-SP levels in the range of from three to seven times higher than an ANP-SP control level.

47. A use of ANP-SP binding agent for the prediction, diagnosis or monitoring of acute cardiac disorder (ACD), cardiac transplant rejection or ACD/pulmonary disorder in a subject, wherein the prognosis, diagnosis or monitoring is carried out within four hours of onset of or within four hours of clinical presentation with ACD, cardiac transplant rejection or ACD/pulmonary disorder.

48. A use of claim 47 wherein the prediction, diagnosis or monitoring is effected using a method as claimed in any one of claims 5 to 30 or an assay as claimed in any one of claims 31 to 39.

49. A use of any one of claims 44 to 48 wherein the binding agent is a binding agent that binds or selectively binds ANP-SP (SEQ ID NO:14) or a fragment or variant thereof.

50. A use of claim 49 wherein the binding agent is an antibody or antigen-binding fragment thereof.

51. A use of claim 50 wherein the antibody or antigen-binding fragment is an antibody or antigen-binding fragment as claimed in any one of claims 1 to 4.

52. A use of an ANP-SP antibody or antigen-binding fragment thereof as claimed in any one of claims 1 to 4 in the manufacture of a prognostic; diagnostic or monitoring tool for assessing an acute cardiac disorder (ACD), cardiac transplant rejection or ACD/pulmonary disorder in a subject.

53. A use of an ANP-SP antibody or antigen-binding fragment thereof as claimed in any one of claims 1 to 4 for the prediction, diagnosis or monitoring of acute cardiac disorder (ACD), cardiac transplant rejection or ACD/pulmonary disorder in a subject.

54. A kit for predicting, diagnosing or monitoring an acute cardiac disorder (ACD), cardiac transplant rejection or ACD/pulmonary disorder comprising a ANP-SP binding agent as defined in any one of claims 40 to 43, wherein the kit is for use with a biological sample obtained from a subject within four hours of onset of, or clinical presentation with ACD, cardiac transplant rejection or ACD/pulmonary disorder.

55. A kit for predicting, diagnosing or monitoring an acute cardiac disorder (ACD), cardia transplant rejection or ACD/pulmonary disorder comprising a binding agent as defined in any one of claims 40 to 43 wherein the kit is calibrated to measure ANP-SP levels in the range of 0.1 to 500 pmol/L, preferably 1 to 300 pmol/L, and preferably 2 to 100 pmol/L.

56. A kit for predicting, diagnosing or monitoring an acute cardiac disorder (ACD), cardiac transplant rejection or ACD/pulmonary disorder comprising a ANP-SP antibody or antigen-binding fragment thereof as claimed in any one of claims 1 to 4.

57. A kit as claimed in any one of claims 54 to 56 which further comprises a solid phase on which the binding agent or antibody is immobilised.

58. A kit as claimed in any one of claims 54 to 57 which further comprises instructions for predicting, diagnosing or monitoring ACD, cardiac transplant rejection, or ACD/pulmonary disorder in a subject within four hours of onset, or clinical presentation, from the ANP-SP level measured in the biological sample obtained within four hours of onset or clinical presentation.

59. A nucleic acid molecule encoding ANP-SP (16-25) (SEQ ID NO:12) or ANP-SP (1-10) (SEQ ID NO:16) or a fragment or variant thereof wherein said nucleic acid is
(a) SEQ ID NO:13 or SEQ ID NO:17 or a variant or fragment thereof;
(b) a sequence which has 70%, 75%, 80%, 90%, 95% or 99% sequence identity to SEQ ID NO:13 or SEQ ID NO:17;
(c) a sequence which hybridises under stringent conditions to SEQ ID NO:13 or SEQ ID NO:17 or a fragment or variant thereof;
(d) a sequence of at least 10 nucleotides in length, capable of hybridising to the sequence of any one of (a) to (c) under stringent conditions;
(e) a complement of any one of (a) to (d);
with the proviso that the sequence is not SEQ ID NO: 15.

60. A genetic construct which comprises a nucleic acid molecule of claim 59.

61. A genetic construct of claim 60 which is an expression construct.

62. A vector which comprises a genetic construct of claim 60 or claim 61.

63. A host cell which comprises a genetic construct or vector according to any one of claims 60 to 62.

64. An ANP-SP polypeptide encoded by a nucleic acid molecule of claim 59, or a variant or fragment thereof.

65. An ANP-SP polypeptide or a variant or fragment thereof selected from:
(a) ANP-SP (16-25) (SEQ ID NO:12) or a variant or fragment thereof;
(b) ANP-SP (1-10) (SEQ ID NO: 16) or a variant or fragment thereof; or
(c) an amino acid sequence having at least 70%, 75%, 80%, 85%, 90%, 95% or 99% amino acid identity to the polypeptide of SEQ ID NO:12 or SEQ ID NO:16.

66. A method for the recombinant production of a polypeptide according to claim 64 or claim 65, the method comprising the steps of:
(a) culturing a host cell comprising a genetic construct of claim 60 or claim 61 capable of expressing a polypeptide of claim 64 or claim 65; and
(b) selecting cells expressing the polypeptide of the invention;
(c) separating the expressed polypeptide from the cells; and optionally
(d) purifying the expressed polypeptide.

67. A method of claim 66 wherein the method comprises a pre-step of transfecting the host cells with the construct.


SEQUENCE LISTING (17 sequences)

SEQ ID NO: 1 - protein, 151 aa, Homo sapiens
  ref <308>: DATABASE ACCESSION NUMBER: NP_006163
  ref <309>: DATABASE ENTRY DATE: 2008-02-17
  ref <313>: RELEVANT RESIDUES IN SEQ ID NO: (1)..(151)
MSSFSTTTVSFLLLLAFQLLGQTRANPMYNAVSNADLMDFKNLLDHLEEKMPLEDEVVPPQVLSEPNEEAGAALSPLPEVPPWTGEVSPAQRDGGALGRGPWDSSDRSALLKSKLRALLTAPRSLRRSSCFGGRMDRIGAQSGLGCNSFRY

SEQ ID NO: 2 - DNA, 840 bp, Homo sapiens
  ref <308>: DATABASE ACCESSION NUMBER: NM_006172
  ref <309>: DATABASE ENTRY DATE: 2008-02-17
  ref <313>: RELEVANT RESIDUES IN SEQ ID NO: (1)..(840)
agggacagacgtaggccaagagaggggaaccagagaggaaccagaggggagagacagagcagcaagcagtggattgctccttgacgacgccagcatgagctccttctccaccaccaccgtgagcttcctccttttactggcattccagctcctaggtcagaccagagctaatcccatgtacaatgccgtgtccaacgcagacctgatggatttcaagaatttgctggaccatttggaagaaaagatgcctttagaagatgaggtcgtgcccccacaagtgctcagtgagccgaatgaagaagcgggggctgctctcagccccctccctgaggtgcctccctggaccggggaagtcagcccagcccagagagatggaggtgccctcgggcggggcccctgggactcctctgatcgatctgccctcctaaaaagcaagctgagggcgctgctcactgcccctcggagcctgcggagatccagctgcttcgggggcaggatggacaggattggagcccagagcggactgggctgtaacagcttccggtactgaagataacagccagggaggacaagcagggctgggcctagggacagactgcaagaggctcctgtcccctggggtctctgctgcatttgtgtcatcttgttgccatggagttgtgatcatcccatctaagctgcagcttcctgtcaacacttctcacatcttatgctaactgtagataaagtggtttgatggtgacttcctcgcctctcccaccccatgcattaaattttaaggtagaacctcacctgttactgaaagtggtttgaaagtgaataaacttcagcaccatggac

SEQ ID NO: 3 - protein, 152 aa, Rattus norvegicus
  ref <308>: DATABASE ACCESSION NUMBER: NP_036744
  ref <309>: DATABASE ENTRY DATE: 2008-02-11
  ref <313>: RELEVANT RESIDUES IN SEQ ID NO: (1)..(152)
MGSFSITKGFFLFLAFWLPGHIGANPVYSAVSNTDLMDFKNLLDHLEEKMPVEDEVMPPQALSEQTDEAGAALSSLSEVPPWTGEVNPSQRDGGALGRGPWDPSDRSALLKSKLRALLAGPRSLRRSSCFGGRIDRIGAQSGLGCNSFRYRR

SEQ ID NO: 4 - DNA, 831 bp, Rattus norvegicus
  ref <308>: DATABASE ACCESSION NUMBER: NM_012612
  ref <309>: DATABASE ENTRY DATE: 2008-02-11
  ref <313>: RELEVANT RESIDUES IN SEQ ID NO: (1)..(831)
cggacaaaggctgagagagaaaccagagagtgagccgagacagcaaacatcagatcgtgccccgacccacgccagcatgggctccttctccatcaccaagggcttcttcctcttcctggccttttggctcccaggccatattggagcaaatcccgtatacagtgcggtgtccaacacagatctgatggatttcaagaacctgctagaccacctggaggagaagatgccggtagaagatgaggtcatgcctccgcaggccctgagcgagcagaccgatgaagcgggggcggcacttagctccctctctgaggtgcctccctggactggggaagtcaacccgtctcagagagatggaggtgctctcgggcgcggcccctgggacccctccgatagatctgccctcttgaaaagcaaactgagggctctgctcgctggccctcggagcctgcgaaggtcaagctgcttcgggggtaggattgacaggattggagcccagagcggactaggctgcaacagcttccggtaccgaagataacagccaaatctgctcgagcagatcgcaaaagatcccaagcccttgcggtgtgtcacacagcttggtcgcattgccactgagaggtggtgaataccctcctggagctgcagcttcctgtcttcatctatcacgatcgatgttaagtgtagatgagtggtttagtgaggccttacctctcccactctgcatattaaggtagatcctcacccctttcagaaagcagttggaaaaaaataaatccgaataaacttcagcaccacggacagacgctgaggcctgaaaaaaaaaaaaaaaaa

SEQ ID NO: 5 - protein, 152 aa, Ovis aries
  ref <308>: DATABASE ACCESSION NUMBER: AAB92564
  ref <309>: DATABASE ENTRY DATE: 2001-02-05
  ref <313>: RELEVANT RESIDUES IN SEQ ID NO: (1)..(152)
MGSSAITTSFLLFVAFQLPGQTGANPVYGSVSNADLMDFKNLLDRLEDKMPLEDEAVPSQVLSEQNEEAGAPLSPLSEVPPWDGGRSTQPREMGAPSDGDPGNPPRSVLLKSKLRALLTAPRSLRRSSCFGGRMDRIGAQSGLGCNSFRYRR

SEQ ID NO: 6 - DNA, 2207 bp, Ovis aries
  ref <308>: DATABASE ACCESSION NUMBER: AFO37465
  ref <309>: DATABASE ENTRY DATE: 2001-02-05
  ref <313>: RELEVANT RESIDUES IN SEQ ID NO: (1)..(2207)
tgaattaggactctagaaaactactttctcttcacctggacactgagagggacagtcagggggtgggggtagccctgcggggggcaggttctgccctccgccactgctgatgagtcaggtgtgaggccagctccagcatctggactaatttgtccgatgccgagcagccggctgcctgccacatcctcccctcccgcccttatttggagctgacagctgagcagcaaaccaacaggggagctgggcgcccgccaaccgtcaccctctgcttccccgcatgagtcccgttgccgaggagaaagaagccggaggcatcagtgtgagataaccaaggactcttttttgctcttctcacaccttgaagtgggaacctcttgaggcaaatcaacaagaatgtggctcctgcagctgagggtcctgggggttgtcggggctgctcaaggcagaggggctgtgacaagcaggctggactgataactttaaaaatcttctgctgcttcctcactcagctgctttatcactgcaagtgacaggatggggagggttccagctctccggacgagctcccagagagccagggggctataaaaagaggaggctcagggcagctgggagacagagacggacaaaggccaacagtgaaaggccaaagaagccagagaggaggcagcaagcaccagaccgaccattccttgaccgaagccagggggctataaaaagaggaggctcagggcagctgggagacagagacggacaaaggccaacagtgaaaggccaaagaagccagagaggaggcagcaagcaccagaccgaccattccttgaccgacgccagcatgggctcctccgccatcaccacgagcttcctcctctttgtggcgtttcagctcccagggcaaacaggagcaaatcccgtgtatggctctgtgtccaatgcagacctgatggatttcaaggtagggccaggaaagcggcatggtctggggtgagggggttgtgacattgggccaggcagcgagacttctccctttccattttccttttgtaaagaatttgctggaccgtttggaggacaagatgcctttagaagatgaggctgtgccctcacaagtattaagtgagcagaatgaagaagctggggcccctctcagccctctttcagaggtgcctccctgggatggggggaggtcaacgcagcccagagagatgggggcgccctcggacggggaccctgggaatcctccgagatctgtcctcctgaagagcaagctgagggcactgctcactgcccctcggagcctgcggaggtccagctgcttcgggggaaggatggacaggattggagcccagagtggattgggctgcaacagcttccgggtaagaggacctgaggatggaagtgggatggggaggaaggaaattatggtttcattgacattcaatccttgcgaaagaacaccacccgggaatgccttcagtaggaaagggacagcatagaagcaacccctttaaaatttctgccccaaactggcggggagggggttgtgctctgagtctcaggacaatgataccaacctcagctacagttggctgagaaaatgctaagaaaaaaaaactttactgccacgagcaatggggacttaaattgttcatggggccaaatcacctgtgctctgctggttggtagttcatgtcctttgcagaatcatcagattccaaaggattgaaattgggcaggactgactttactagctcctaacgggcaatttgtttaccagtttacagaagtcagagggtcatcaggctggagtggaggctggtgggaagggagcagagtctgatgaagctgacttttccggtggagtcaggtcaccaaaccaaacatgtctctgctctcttgtagtatcgaagataacggccagggaggaggaaaaggcaggccaggccccaggcagtcttcaagagaatcccctggggtctctcactcaactttgtcgcatctggttgccatcaagttgagttgtgacaagtgttctattcaagcatcagcttcctgtcaacatttctcacattttatgctaaatgtagacaaagtgatttaactgtggcttctccacctctcccacccatgtgttaagtttttatcacctgttaccaacatcagtttgaaaatgaataaacttcacaccatggacagaaacagtaggc

SEQ ID NO: 7 - protein, 150 aa, Sus scrofa
  ref <308>: DATABASE ACCESSION NUMBER: NP_999425
  ref <309>: DATABASE ENTRY DATE: 2007-09-25
  ref <313>: RELEVANT RESIDUES IN SEQ ID NO: (1)..(150)
MSSFTITVSFLLVLVFQFPGQTRANPVYGSVSNADLMDFKNLLDHLEDKMPLEDEAMPPQVLSEQNEEVGAPLSPLLEVPPWTGEVNPAQRDGGALGRGPWDASDRSALLKSKLRALLAAPRSLRRSSCFGGRMDRIGAQSGLGCNSFRY

SEQ ID NO: 8 - DNA, 474 bp, Sus scrofa
  ref <308>: DATABASE ACCESSION NUMBER: NM_214260
  ref <309>: DATABASE ENTRY DATE: 2007-09-25
  ref <313>: RELEVANT RESIDUES IN SEQ ID NO: (1)..(474)
acgacgccagcatgagctccttcaccatcaccgtgagcttcctcctcgttctggtgtttcagttcccagggcaaaccagagcgaaccctgtgtacggctccgtgtccaatgcagacctgatggatttcaagaatttgctggaccacttggaggacaagatgcctttagaagatgaggctatgcccccacaagtactaagcgagcagaatgaggaagtgggggcccctctcagcccccttttggaggtacctccctggaccggggaggtgaacccagcccagagagatgggggtgccctggggcggggcccctgggacgcttccgatagatctgcccttctgaagagcaagctgagggcactgcttgctgcccctcggagcctgcggaggtccagctgcttcgggggtaggatggacaggattggagcacagagtggactgggctgtaacagcttccggtactgaagataacagc

SEQ ID NO: 9 - protein, 152 aa, Mus musculus
  ref <308>: DATABASE ACCESSION NUMBER: NP_032751
  ref <309>: DATABASE ENTRY DATE: 2008-02-10
  ref <313>: RELEVANT RESIDUES IN SEQ ID NO: (1)..(152)
MGSFSITLGFFLVLAFWLPGHIGANPVYSAVSNTDLMDFKNLLDHLEEKMPVEDEVMPPQALSEQTEEAGAALSSLPEVPPWTGEVNPPLRDGSALGRSPWDPSDRSALLKSKLRALLAGPRSLRRSSCFGGRIDRIGAQSGLGCNSFRYRR

SEQ ID NO: 10 - DNA, 844 bp, Mus musculus
  ref <308>: DATABASE ACCESSION NUMBER: NM_008725
  ref <309>: DATABASE ENTRY DATE: 2008-02-10
  ref <313>: RELEVANT RESIDUES IN SEQ ID NO: (1)..(844)
agtgacggacaaaagctgagagagagagagaaagaaaccagagtgggcagagacagcaaacatcagatcgtgccccgacccacgccagcatgggctccttctccatcaccctgggcttcttcctcgtcttggccttttggcttccaggccatattggagcaaatcctgtgtacagtgcggtgtccaacacagatctgatggatttcaagaacctgctagaccacctggaggagaagatgccggtagaagatgaggtcatgcccccgcaggccctgagtgagcagactgaggaagcaggggccgcacttagctccctccccgaggtgcctccctggactggggaggtcaacccacctctgagagacggcagtgctctagggcgcagcccctgggacccctccgatagatctgccctcttgaaaagcaaactgagggctctgctcgctggccctcggagcctacgaagatccagctgcttcgggggtaggattgacaggattggagcccagagtggactaggctgcaacagcttccggtaccgaagataacagccaaggaggaaaaggcagtcgattctgcttgagcagatcgcaaaagatcctaagcccttgtggtgtgtcacgcagcttggtcacattgccactgtggcgtggtgaacaccctcctggagctgcggcttcctgccttcatctatcacgatcgatgttaaatgtagatgagtggtctagtggggtcttgcctctcccactctgcatattaaggtagatcctcacccttttcagaaagcagttggaaaaaaaaaaaaagaataaacttcagcaccaaggacgaaaaaaaaaaaaaa

SEQ ID NO: 11 - protein, 149 aa, Canis familiaris
  ref <308>: DATABASE ACCESSION NUMBER: P07499 Uniprot
  ref <309>: DATABASE ENTRY DATE: 2007-10-23
  ref <313>: RELEVANT RESIDUES IN SEQ ID NO: (1)..(149)
MGSPIAASFLLFLAVQLLGQTGANPVYGSVSNADLLDFKNLLDRLEDKMPLEDEAESPQALSEQNAEAGAALSPLPEVPPWTGEVSPAQRDGGALGRSPWDSSDRSALLKSKLRALLAAPRSLRRSSCFGGRMDRIGAQSGLGCNSFRY

SEQ ID NO: 12 - protein, 10 aa, Homo sapiens
  ref <308>: DATABASE ACCESSION NUMBER: NP_006163
  ref <309>: DATABASE ENTRY DATE: 2008-02-17
  ref <313>: RELEVANT RESIDUES IN SEQ ID NO: (16)..(25)
AFQLLGQTRA

SEQ ID NO: 13 - DNA, 30 bp, Homo sapiens
  ref <308>: DATABASE ACCESSION NUMBER: NM_006172
  ref <309>: DATABASE ENTRY DATE: 2008-02-17
  ref <313>: RELEVANT RESIDUES IN SEQ ID NO: (140)..(169)
gcattccagctcctaggtcagaccagagct

SEQ ID NO: 14 - protein, 25 aa, Homo sapiens
  ref <308>: DATABASE ACCESSION NUMBER: NP_006163
  ref <309>: DATABASE ENTRY DATE: 2008-02-17
  ref <313>: RELEVANT RESIDUES IN SEQ ID NO: (1)..(25)
MSSFSTTTVSFLLLLAFQLLGQTRA

SEQ ID NO: 15 - DNA, 75 bp, Homo sapiens
  ref <308>: DATABASE ACCESSION NUMBER: NM_006172
  ref <309>: DATABASE ENTRY DATE: 2008-02-17
  ref <313>: RELEVANT RESIDUES IN SEQ ID NO: (95)..(169)
atgagctccttctccaccaccaccgtgagcttcctccttttactggcattccagctcctaggtcagaccagagct

SEQ ID NO: 16 - protein, 10 aa, Homo sapiens
  ref <308>: DATABASE ACCESSION NUMBER: NP_006163
  ref <309>: DATABASE ENTRY DATE: 2008-02-17
  ref <313>: RELEVANT RESIDUES IN SEQ ID NO: (1)..(10)
MSSFSTTTVS

SEQ ID NO: 17 - DNA, 30 bp, Homo sapiens
  ref <308>: DATABASE ACCESSION NUMBER: NM_006172
  ref <309>: DATABASE ENTRY DATE: 2008-02-17
  ref <313>: RELEVANT RESIDUES IN SEQ ID NO: (95)..(124)
atgagctccttctccaccaccaccgtgagc